(19) United States
(12) Patent Application Publication
(10) Pub. No.: US 2022/0218244 A1
(43) Pub. Date: Jul. 14, 2022

(54) WEARABLE PULSE OXIMETER FOR TENNIS PLAYERS

(71) Applicant: Masimo Corporation, Irvine, CA (US)

(72) Inventors: Massi Joe E. Kiani, Laguna Niguel, CA (US); Bilal Muhsin, Irvine, CA (US)

(21) Appl. No.: 17/572,525

(22) Filed: Jan. 10, 2022

Related U.S. Application Data
(60) Provisional application No. 63/136,150, filed on Jan. 11, 2021.

(51) Int. Cl. A61B 5/1455; A61B 5/024; A61B 5/00

(52) U.S. Cl. CPC A61B 5/14551; A61B 5/024; A61B 5/681; A61B 2503/10; A61B 5/743; A61B 5/744; A61B 5/746

(57) ABSTRACT

The present disclosure is directed to methods and systems for combining physiological data from a pulse oximeter coupled to a player playing a tennis match with match data corresponding to the tennis match. An example system can comprise an emitter configured to transmit optical radiation towards a tissue site of the player; a detector configured to detect light attenuated from the tissue site responsive to the transmitted optical radiation; and one or more hardware processors. The one or more hardware processors can be configured, via executable software instructions, to: receive physiological data responsive to the detected light, wherein the physiological data can include one or more physiological parameters including a pulse rate; receive match data corresponding to an occurrence in the tennis match; and generate a visual alert to one or more viewers of the tennis match based on a trigger event corresponding to the received physiological data and the match data.

### CROSS REFERENCE TO RELATED APPLICATIONS

This application claims benefit of priority to U.S. Provisional Patent Application No. 63/136,150, filed Jan. 11, 2021.

### FIELD OF THE DISCLOSURE

The present disclosure relates to gathering physiological data from event participants using physiological sensors during events such as sports events and displaying the physiological data.

### BACKGROUND

Physiological sensors can be used to gather data from a subject. The data can be processed or analyzed to provide information, such as physiological parameters, relating to a physiology of the subject. Events, such as sports events, are often viewed by spectators or fans. Viewers can view the event via a screen. The screen may be part of a device and may be remote to the event or located at the event.

### SUMMARY

Various embodiments of systems, methods and devices within the scope of the appended claims each have several aspects, no single one of which is solely responsible for the desirable attributes described herein. Without limiting the scope of the appended claims, the description below describes some prominent features.

Details of one or more embodiments of the subject matter described in this specification are set forth in the accompanying drawings and the description below. Other features, aspects, and advantages will become apparent from the description, the drawings, and the claims. Note that relative dimensions of the following figures may not be drawn to scale.

The present disclosure provides a system for providing additional data about participants of an event. The system may comprising: one or more hardware processors configured, via executable software instructions, to: receive physiological data from one or more physiological sensors coupled to an event participant, wherein the physiological data may include one or more physiological parameters; receive event data corresponding to an occurrence at the event; generate visual display data for rendering one or more visual displays, wherein the one or more visual displays can be based, at least, on the physiological data and the event data, and wherein the one or more visual displays can include at least one of the one or more physiological parameters; and transmit the visual display data to a display for displaying the one or more visual displays.

In some embodiments, the event data can be received from a database.

In some embodiments, the event data is received via manual input.

In some embodiments, the display can be configured to display, concurrently, the one or more visual displays and a graphical representation of the event.

In some embodiments, the display can be located at the event.

In some embodiments, the display can be located remote to the event.

In some embodiments, the event can be a sports event.

In some embodiments, the event can be a tennis match.

In some embodiments, the event can be a video game event.

In some embodiments, the video game event can be a competition or tournament.

In some embodiments, the video game can be a first-person game, a first-person shooter (FPS) game, a role-playing (RPG) game, a real-time strategy (RTS) game, a massively multiplayer online game, a massively multiplayer online role-playing (MMORPG) game, an exploring game, an action game, a simulation game, a strategy game, a sports game, a puzzle game, or a multiplayer online battle arena game.

In some embodiments, the event can be a musical or dance or theater performance.

In some embodiments, the event participants can be athletes.

In some embodiments, the event participants can be players.

In some embodiments, the event participants can be tennis players.

In some embodiments, the event participants can be video game players.

In some embodiments, the event participants can be animals.

In some embodiments, the event data can include one or more of an event score or an event time or a time.

In some embodiments, the event data can include statistics of event participants, including one or more of participants points, participant fouls, participant errors, or participant playing time.

In some embodiments, the statistics can include statistics of the event or statistics of one or more previous events.

In some embodiments, the one or more physiological parameters can include one or more of heart rate, pulse rate, SpO2, respiration rate, ECG, hemoglobin concentration or amount, or body temperature.

In some embodiments, the one or more hardware processors is further configured to synchronize the physiological data with the event data.

The present disclosure provides a system for providing additional data about participants of an event. The system can comprise: one or more hardware processors configured, via executable software instructions, to: receive physiological data from one or more physiological sensors coupled to an event participant, wherein the physiological data can include one or more physiological parameters; receive event data corresponding to an occurrence at the event; generate visual display data for rendering one or more visual displays, wherein the one or more visual displays can be based, at least, on the physiological data and the event data, and wherein the visual display can provide an indication of the mental state or physiological state of the event participant to explain participant performance.

In some embodiments, the visual display can include a graphical representation relating to the physiological data or to at least one of the one or more physiological parameters.

In some embodiments, the graphical representation can be an ECG waveform.

In some embodiments, the graphical representation can be a heart.

In some embodiments, the one or more visual displays can include an avatar representation of the event participant.

In some embodiments, a color of the avatar can be based on at least one of the one or more physiological parameters, and the avatar can be configured to change color in response to a change in value of at least one of the one or more physiological parameters.

In some embodiments, the avatar can be red when a physiological parameter relating to temperate exceeds a threshold.

In some embodiments, the avatar can be configured to perform an action, wherein the action is based on at least one of the one or more physiological parameters.

In some embodiments, the one or more visual displays can include a graph or chart of at least one of the one or more physiological parameters.

In some embodiments, the graph or chart can be a line graph, bar chart, scatter plot, 3D graph, or pie chart.

In some embodiments, the one or more visual displays can include a trend of at least one of the one or more physiological parameters.

In some embodiments, the visual display data can include data relating to a portion of a screen in which to render the visual display.

The present disclosure provides a system for providing additional data about participants of an event. The system may comprise: one or more hardware processors configured, via executable software instructions, to: receive first physiological data from one or more first physiological sensors coupled to a first event participant, wherein the first physiological data can include one or more first physiological parameters; receive second physiological data from one or more second physiological sensors coupled to a second event participant, wherein the second physiological data can include one or more second physiological parameters; and generate based, at least, on the first physiological data and the second physiological data, visual display data for rendering one or more visual displays to provide a visual indication of the mental state or physiological state of the first and second event participants.

In some embodiments, the one or more visual displays can include a first trend of at least one of the one or more first physiological parameters and a second trend of at least one of the one or more second physiological parameters.

In some embodiments, the first and second trends can be overlaid on a graph to provide a visual comparison of the physiological states of the first and second event participants.

In some embodiments, the first and second trends can correspond to a time elapsed during the event.

The present disclosure provides a system for providing additional data about participants of an event. The system may comprise: one or more hardware processors configured, via executable software instructions, to: receive physiological data from one or more physiological sensors coupled to an event participant, wherein the physiological data can include one or more physiological parameters; receive historical physiological data of the event participant, wherein the historical physiological data may correspond to physiological data gathered from the event participant during one or more previous events in which the event participant has participated, and wherein the historical physiological data can include one or more historical physiological parameters; and generate based, at least, on the physiological data and the historical physiological data, visual display data for rendering one or more visual displays to provide a visual indication of the mental state or physiological state of the event participant.

In some embodiments, the one or more visual displays can include a first trend of at least one of the one or more physiological parameters and a second trend of at least one of the one or more historical physiological parameters.

In some embodiments, the first and second trends can be overlaid on a graph to provide a visual comparison of the physiological states of the event participant during the event and during the one or more previous events.

The present disclosure provides a system for providing additional data about participants of an event. The system may comprise: one or more hardware processors configured, via executable software instructions, to: receive physiological data from one or more physiological sensors coupled to an event participant, wherein the physiological data can include one or more physiological parameters; receive event data corresponding to an occurrence at the event; store, in a database, the physiological data as historical physiological data; store, in the database, the event data as historical event data; and generate visual display data for rendering one or more visual displays, wherein the one or more visual displays can be based, at least, on the physiological data and the event data.

In some embodiments, the one or more hardware processors can be further configured to: access the database to retrieve the historical physiological data, and the one or more visual displays can be based, at least, on the historical physiological data.

In some embodiments, the one or more hardware processors can be further configured to: access the database to retrieve the historical event data, and the one or more visual displays can be based, at least, on the historical event data.

The present disclosure provides a system for providing additional data about participants of an event. The system may comprise: one or more hardware processors configured, via executable software instructions, to: receive physiological data from one or more physiological sensors coupled to an event participant, wherein the physiological data can include one or more physiological parameters; receive event data corresponding to an occurrence at the event; and generate visual display data for rendering one or more visual displays in response to the occurrence of a physiological condition of the event participant or in response to the occurrence of an event condition of the event, wherein the physiological condition can be determined based, at least, on the physiological data, and wherein the event condition can be determined based, at least, on the event data.

In some embodiments, the event condition is a time out, a break, a change in score, an elapsed time, a commencement of the event, or a termination of the event.

In some embodiments, the event condition can occur when a score exceeds a threshold.

In some embodiments, the event condition can occur when a difference between scores falls below a threshold.

In some embodiments, the event condition can occur when a time remaining in the event falls below a threshold.

In some embodiments, the physiological condition can be a change in value of at least one of the one or more physiological parameters, wherein the change in value exceeds a threshold.

In some embodiments, the one or more hardware processors can be configured to: generate the visual display data in response to a request.

In some embodiments, the request can be a user selection via the display.

In some embodiments, the one or more hardware processors can be configured to: generate, in response to a user selection, updated visual display data for rendering an updated visual display.

The present disclosure provides a system for providing additional data about participants of an event. The system may comprise: one or more hardware processors configured, via executable software instructions, to: receive physiological data from one or more physiological sensors coupled to an event participant, wherein the physiological data can include one or more physiological parameters; receive event data corresponding to an occurrence at the event; determine, based, at least, on the physiological data and the event data, a future occurrence; and determine, based, at least, on the physiological data and the event data, a probability that the future occurrence will occur.

In some embodiments, the future occurrence can be a final event score, a change in event score, a participant ranking, an event outcome, an event winner, or an event loser.

In some embodiments, the future occurrence can be a participant action, including at least one of scoring a point, winning an event, losing an event, breaking a record, taking a break, or making a mistake or error.

The present disclosure provides a method for providing additional data about participants of an event. The method may comprise: receiving physiological data from one or more physiological sensors coupled to an event participant, wherein the physiological data includes one or more physiological parameters; receiving event data corresponding to an occurrence at the event; generating visual display data for rendering one or more visual displays, wherein the one or more visual displays is based, at least, on the physiological data and the event data, and wherein the one or more visual displays includes at least one of the one or more physiological parameters; and transmitting the visual display data to a display for displaying the one or more visual displays.

The present disclosure provides a method for providing additional data about participants of an event. The method may comprise: receiving physiological data from one or more physiological sensors coupled to an event participant, wherein the physiological data includes one or more physiological parameters; receiving event data corresponding to an occurrence at the event; generating visual display data for rendering one or more visual displays, wherein the one or more visual displays is based, at least, on the physiological data and the event data, and wherein the visual display provides an indication of the mental state or physiological state of the event participant to explain participant performance.

The present disclosure provides a method for providing additional data about participants of an event. The method may comprise: receiving first physiological data from one or more first physiological sensors coupled to a first event participant, wherein the first physiological data includes one or more first physiological parameters; receiving second physiological data from one or more second physiological sensors coupled to a second event participant, wherein the second physiological data includes one or more second physiological parameters; and generating based, at least, on the first physiological data and the second physiological data, visual display data for rendering one or more visual displays to provide a visual indication of the mental state or physiological state of the first and second event participants.

The present disclosure provides a method for providing additional data about participants of an event. The method may comprise: receiving physiological data from one or more physiological sensors coupled to an event participant, wherein the physiological data includes one or more physiological parameters; receiving historical physiological data of the event participant, wherein the historical physiological data corresponds to physiological data gathered from the event participant during one or more previous events in which the event participant has participated, and wherein the historical physiological data includes one or more historical physiological parameters; and generating based, at least, on the physiological data and the historical physiological data, visual display data for rendering one or more visual displays to provide a visual indication of the mental state or physiological state of the event participant.

The present disclosure provides a method for providing additional data about participants of an event. The method may comprise: receiving physiological data from one or more physiological sensors coupled to an event participant, wherein the physiological data includes one or more physiological parameters; receiving event data corresponding to an occurrence at the event; storing, in a database, the physiological data as historical physiological data; storing, in the database, the event data as historical event data; and generating visual display data for rendering one or more visual displays, wherein the one or more visual displays is based, at least, on the physiological data and the event data.

The present disclosure provides a method for providing additional data about participants of an event. The method may comprise: receiving physiological data from one or more physiological sensors coupled to an event participant, wherein the physiological data includes one or more physiological parameters; receiving event data corresponding to an occurrence at the event; and generating visual display data for rendering one or more visual displays in response to the occurrence of a physiological condition of the event participant or in response to the occurrence of an event condition of the event, wherein the physiological condition is determined based, at least, on the physiological data, and wherein the event condition is determined based, at least, on the event data.

The present disclosure provides a method for providing additional data about participants of an event. The method may comprise: receiving physiological data from one or more physiological sensors coupled to an event participant, wherein the physiological data includes one or more physiological parameters; receiving event data corresponding to an occurrence at the event; determining, based, at least, on the physiological data and the event data, a future occurrence; and determining, based, at least, on the physiological data and the event data, a probability that the future occurrence will occur.

### BRIEF DESCRIPTION OF THE DRAWINGS

FIG. 1 illustrates an example display for displaying event related data, participant physiology related data, and the like.

FIGS. 2A-2G illustrate example sensors that may be worn by event participants and which may gather physiological data of the event participants.

FIG. 3 is a block diagram illustrating an example system for gathering and displaying physiological data of event participants.

FIG. 4 is a block diagram illustrating an example controller.

FIG. 5 is a flowchart illustrating an example process for generating display data for displaying event related data and/or participant physiological related data.

FIG. 6 is a flowchart illustrating an example process for predicting participant performance or event outcome.

FIG. 7 is a flowchart illustrating an example process for determining the reliability of data.

FIGS. 8-23 illustrate example displays for displaying event related data, participant physiology related data, and the like.

### DETAILED DESCRIPTION

### Overview

Physiological sensors can be used to gather physiological data, such as oxygen saturation (SpO2) or pulse rate (PR), of an individual. This may be useful in medical settings such as monitoring the physiological data of a patient in a hospital. Physiological sensors can also be used in other settings wherein it may be desirable to view and/or monitor an individual's physiological data. For example, physiological sensors can be used to monitor participants in sports events such as tennis, basketball, surfing, baseball, football, hockey, volleyball, soccer, running, cycling, swimming, climbing, skiing, golf, or other similar events. In some implementations, the event may be a competition, a practice, a scrimmage, a training session, and the like. Physiological sensors can also be used to monitor the physiological data of participants in other events such as dance performances, musical performances, concerts, chess tournaments, racing events such as NASCAR or horse races. Physiological sensors can also be used to monitor the physiological data of participants in video game related events such as video game tournaments or competitions including video games such as a first-person game, a first-person shooter (FPS) game, a role-playing (RPG) game, a real-time strategy (RTS) game, a massively multiplayer online game, a massively multiplayer online role-playing (MMORPG) game, an exploring game, an action game, a simulation game, a strategy game, a sports game, a puzzle game, or a multiplayer online battle arena game. Physiological sensors can also be used to monitor the physiological data of participants in other events such as political events, for example political rallies or political speeches, public speaking events, educational speeches, lectures, webinars, the production of videos or films, intellectual competitions such as spelling bees, or supervising or monitoring other individuals such as employees or children.

In addition to the various events wherein it may be desirable to monitor an individual's physiological data by use of a physiological sensor, physiological sensors can also be used to monitor a variety of participants in such events and described above. For example, physiological sensors can be used to monitor the physiological data of the players in a sports event, the officials in sports events, such as the referees, the coaches, the managers or the owners, the audience, the spectators, the fans, the viewers, and the like. Additionally, the physiological sensors can be used to monitor the physiological data of humans or non-humans, such as animals, such as the horses in a horse race.

As described above, physiological sensors may be used in many contexts wherein it may be desirable to monitor and/or obtain the physiological data of a person of interest such as a tennis player in a tennis match. This physiological data may be useful for medical/health related purposes or non-medical/health related purposes. For example, the physiological data may be used to provide entertainment to viewers of the event. As another example, the physiological data may be used to provide feedback to an event participant, such as a player, about their performance. As another example, the physiological data may be used by health providers to analyze a player's health and determine the health status of a player such as prior to a sports event to verify a player is healthy to play or during a sports event such as when the player has physically exerted themself or when the player has experienced an injury.

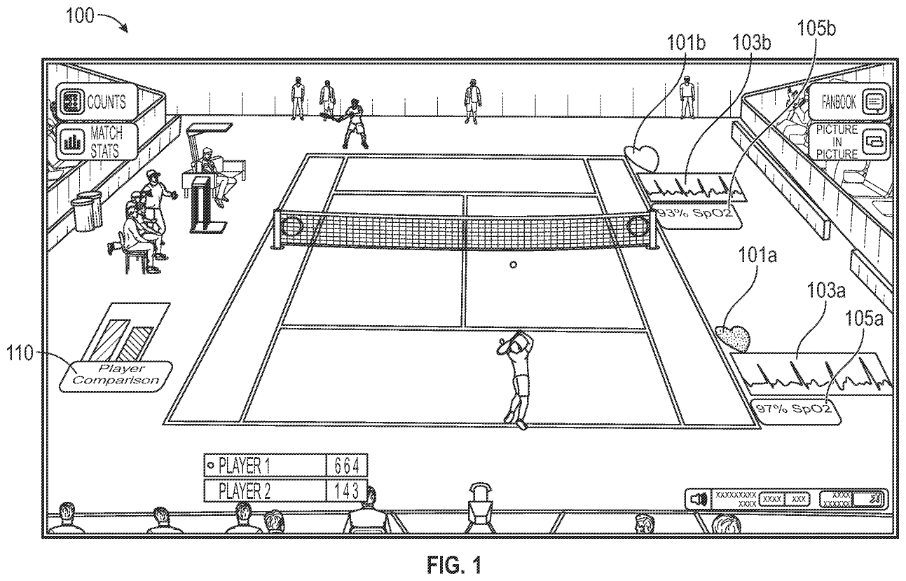

FIG. 1 illustrates an example display 100 that displays an event and corresponding physiological data. In this example, the event is a tennis match and the event participants are the tennis players. Physiological data is gathered from one or more sensors of the tennis players. For example, the tennis players may be wearing a blood oxygen saturation sensor and a cardiac activity sensor. The physiological sensors gather physiological data which can then be processed (e.g., by the sensor or other computing device) to output one or more physiological parameters such as SpO2 or heart rate. The physiological data and/or parameters are communicated to a control system which generates display data for rendering one or more visual displays associated with the tennis match, the physiological data and/or parameters. The display data is rendered by a display device to be viewed by viewers of the tennis match. The physiological data and/or parameters may be displayed in real-time with the actual physiology of the tennis players and/or the events of the tennis match.

As shown, the display 100 includes heart icons 101a, 101b, ECG waveforms 103a, 103b, and SpO2 parameters 105a, 105b. The heart icons 101a, ECG waveforms 103a, and SpO2 parameters 105a, are associated with one of the tennis players (the tennis player nearest to them) while heart icons 101b, ECG waveforms 103b, and SpO2 parameters 105b, are associated with the other tennis player.

The heart icons 101a, 101b may be a color that corresponds to physiological state of the associated tennis player. For example, the heart icons 101a, 101b may change color depending on a heart rate or body temperature of the associated tennis player. In some embodiments, heart icons 101a, 101b may beat or pulse. The beating or pulsing of the heart icons 101a, 101b may reflect a real-time heart rate of the associated tennis player. In some embodiments, the heart icons 101a, 101b may be static or motionless.

The ECG waveforms 103a, 103b may reflect real-time cardiac activity of the associated tennis player. The SpO2 parameters 105a, 105b may reflect a real-time blood oxygen saturation of the associated tennis player.

The display 100 includes a player comparison chart 110. The chart 110 may compare various indices, parameters, metrics of the tennis players. For example, the chart 110 may compare a physiological parameter of the tennis players. As another example, the chart 110 may compare an overall index (e.g., health index, mental index), of the tennis players which may be based on averages, combinations, scores etc. of physiological data and/or parameters of the tennis players.

In this example, the heart icons 101a, 101b, ECG waveforms 103a, 103b, SpO2 parameters 105a, 105b, and chart 110 are displayed within the display 100 as superimposed on a ground surface of the tennis court. For example, display data may be generated using green screen techniques using a background of uniform color (e.g., tennis court ground) to display superimposed images as if they were actually on the background. In this example, if an object such as a tennis player were to walk on the ground on a location over which the ECG waveform 101a were displayed, for example, a viewer would view the tennis player as being in front of the ECG waveform 101a.

### Example Sensors

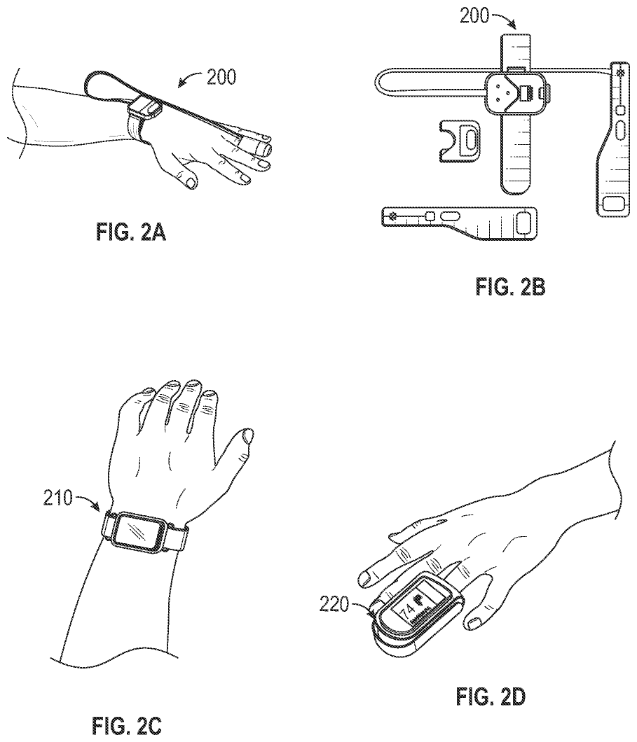

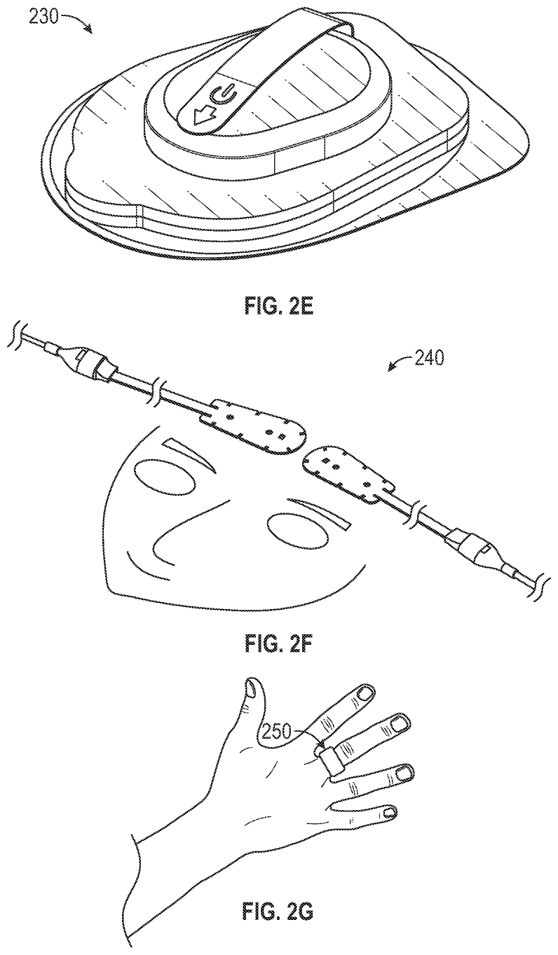

FIGS. 2A-2G illustrate example embodiments of various sensors that may be used to gather physiological data from event participants as described herein. The sensors could include any commercially available sensor from Masimo Corporation of Irvine Calif., or other medical device manufacturer, including but not limited to noninvasive, minimally invasive, or microinvasive glucose sensors, oximetry or cooximetry sensors, pulse rate sensors, cuff and/or continuous noninvasive blood pressure sensors, capnography sensors, acoustic sensors, optical sensors, motion sensors including accelerometers and gyros, pH sensors, image capture sensors using virtually any type of signal and/or wavelength filters, ECG, EEG, depth of sedation, pulse transit time or other parameter responsive to pulse transit time, cardiac parameter sensor, ultrasonic sensor, magnetic imagining sensor, x ray sensor, infrared sensor, proximity sensors, GPS or other location sensors, or the like or combinations thereof. In some embodiments, the sensor can include a detector and an emitter. The detector and the emitter can be optical based. The emitters can include light-emitting diodes (LEDs). In some embodiments, the sensor can generate, using the emitter, an optical output based at least on an emitter signal generated at a processor, and the sensor can detect the optical output using a detector, and convert the optical output to generate raw physiological data.

Additionally, these sensors may be configured to gather a variety of physiological data, such as blood oxygen saturation (SpO2), respiration rate (RR), body temperature, pulse rate or heart rate, cardiac activity, ECG data, perfusion index, pleth variability index, hemoglobin concentration or level, distance travelled, hydration, orientation, heart rate variability, and the like.

As disclosed herein, one or more sensors as shown in FIGS. 1A-1D may be attached to and/or worn by an event participant such as an athlete competing in a sports event. Any number of sensors may be attached to a person of interest, for example, one sensor or more than one sensor. Additionally, the one or more sensors may be attached to various regions of the body for example the head, chest, arm, finger or leg. The sensors may be attached to or worn by the event participant continuously or periodically. For example, the event participant may wear the sensor(s) throughout an entirety of the event or throughout certain durations of the event, for example, while competing in a sports game. As another example, the event participant my only wear the sensor(s) at certain times or intervals throughout the event such as during a time out, break, when not playing while teammates are playing, or halftime etc. The sensor(s) may be integrated with other apparel or gear worn by the event participant. For example, the sensor(s) may be integrated with a headband, wristband, helmet, protective pads, jersey, wristwatch or other wrist worn device, glasses, goggles or any other item worn by or otherwise attached to the event participant during an event.

FIGS. 2A-2B illustrate an example sensor 200 that may be worn on a wrist of an event participant and secured to a digit of the participant. The sensor 200 shown in this example may gather physiological data from the event participant such as heart rate, blood oxygen saturation, perfusion index, pleth variability index, respiration rate, etc. In some embodiments, the sensor 200 is that made commercially available by Masimo Corporation of Irvine, Calif., and marketed under the trademark Radius PPG™.

FIG. 2C illustrates an example sensor 210 that may be worn on a wrist of an event participant. In this example, the sensor 210 is integrated as part a wrist-worn device such as a watch. The sensor 210 shown in this example may gather physiological data from the event participant such as heart rate, SpO2, ECG data etc.

FIG. 2D illustrates an example sensor 220 that may be attached to a digit, such as finger, of an event participant. In this example, the sensor 220 may be attached to an event participant periodically such as to gather data at select intervals during an event such as during a time out or break. The sensor 220 shown in this example may gather physiological data from the event participant such as heart rate, blood oxygen saturation, perfusion index, pleth variability index, respiration rate, etc etc. In some embodiments, the sensor 220 is that made commercially available by Masimo Corporation of Irvine, Calif., and marketed under the trademark Mighty Sat®.

FIG. 2E illustrates an example sensor 230 that may be secured to a body portion of an event participant. For example, the sensor 230 may be secured or affixed to a chest or back of an event participant such as be adhesion. The sensor 230 shown in this example may gather physiological data from the event participant such as body temperature and/or motion data such as orientation or acceleration. In some embodiments, the sensor 230 is that made commercially available by Masimo Corporation of Irvine, Calif., and marketed under the trademark Radius T°™.

FIG. 2F illustrates an example sensor 240 that may be worn on a forehead of an event participant. The sensor 240 shown in this example may gather physiological data from the event participant such as cerebral oxygenation, hemoglobin concentrations or levels or other physiological data relating to the brain. In some embodiments, the sensor 240 is that made commercially available by Masimo Corporation of Irvine, Calif., and marketed under the trademark O3®.

FIG. 2G illustrates an example sensor 250 that may be worn on a digit (such as a finger) of an event participant. The sensor 250 shown in this example may gather physiological data from the event participant such as heart rate, SpO2 etc.

### Example System Implementations

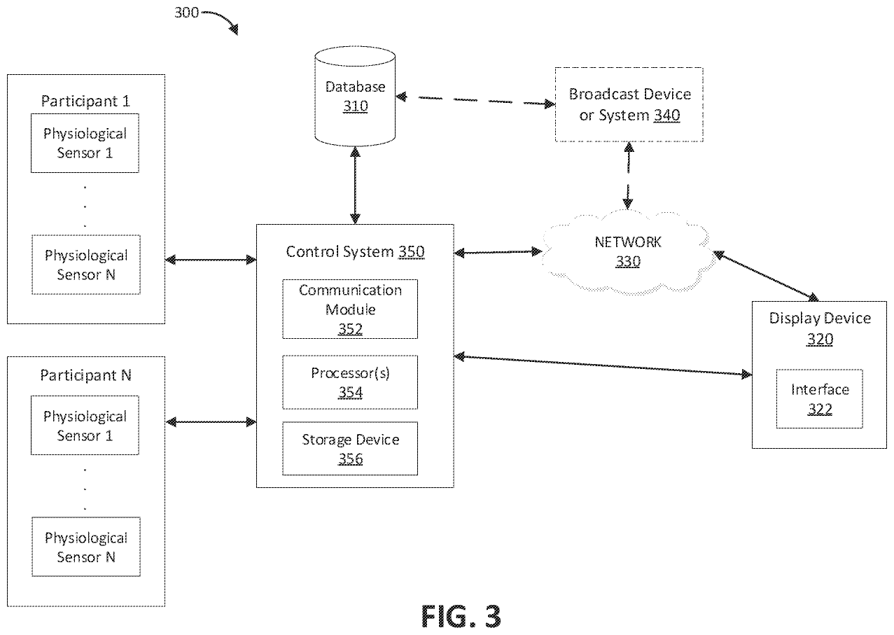

FIG. 3 is a block diagram illustrating an example system 300 for gathering and displaying physiological data of event participants. The system 300 can be implemented in a variety of events such as sports events, video game events, performances, and the like. In some implementations, the system 300 may be implemented during a tennis match.

As shown, the system 300 may include a control system 350, one or more physiological sensors attached or otherwise connected to one or more event participants to gather physiological data from the event participants, a database 310, one or more display devices 320, and a network 330. In some embodiments, the system 300 may include a broadcast device or system 340.

In the example of FIG. 3, the control system 350 includes a communication module 352, one or more processors 354, and a storage device 356. The processor 354 can be configured, among other things, to process data, execute instructions to perform one or more functions, and/or control the operation of the control system 350. For example, the processor 354 can process physiological data obtained from the one or more physiological sensors as well as data received from database 310 and can execute instructions to perform functions related to analyzing, storing, and/or transmitting such data. In some embodiments, the processor 354 can process raw or unprocessed physiological data or signals received from the physiological sensors to derive one or more physiological parameters. In some embodiments, the processor 354 can further process processed physiological data such as physiological parameters received from physiological sensors.

The storage device 356 can include one or more memory devices that store data, including without limitation, dynamic and/or static random access memory (RAM), programmable read-only memory (PROM), erasable programmable read-only memory (EPROM), electrically erasable programmable read-only memory (EEPROM), and the like. The storage device 206 can be configured to store data such as processed and/or unprocessed physiological data obtained from the one or more physiological sensors, event data and the like.

The storage device 356 may be configured to store data that has been transmitted to the control system 350. For example, the storage device 356 can store physiological data received from physiological sensors of the participants, or event related data received from the database 310. Data that may be stored in the storage device 356 may be historical data, such as historical physiological data or historical event related data, because the data that is stored may be transmitted, processed or otherwise used at time that is after (e.g., not in real-time) it has been received by the control system 350. Historical data (e.g., as stored in the storage device 356) may have originated from, and relate to, the event or previous events. The processor 354 can be configured to access the storage device 356 to retrieve the data stored therein.

In some embodiments, data stored in the storage device 356 (and/or the database 310) such as historical physiological data and/or historical event related data may be accessed for subsequent analysis. For example, an event participant's physiological data can be retrieved from the storage device 356 to be analyzed to inform a recovery routine after the event, to aid in training after the event, and the like.

The communication module 352 can facilitate communication (via wired and/or wireless connection) between the control system 350 (and/or components thereof) and separate devices, such as physiological sensors, database 310, the broadcast device or system 340, and display devices 320. For example, the communication module 352 can be configured to allow the control system 350 to wirelessly communicate with other devices, systems, sensors, and/or networks over any of a variety of communication protocols. The communication module 352 can be configured to use any of a variety of wireless communication protocols, such as Wi-Fi (802.11x), Bluetooth®, ZigBee®, Z-wave®, cellular telephony, infrared, near-field communications (NFC), RFID, satellite transmission, proprietary protocols, combinations of the same, and the like. The communication module 352 can allow data and/or instructions to be transmitted and/or received to and/or from the control system 350 and separate computing devices. The communication module 352 can be configured to receive (for example, wirelessly) processed physiological data (such as physiological parameter values) and/or unprocessed physiological data (such as raw sensor signals) from physiological sensors and/or other information such as event related data from database 310 or user inputs from the display device 320. The communication module 352 can be configured to transmit (for example, wirelessly) information such as display information to the display device 320 and/or other separate computing devices, which can include, among others, a mobile device (for example, an iOS or Android enabled smartphone, tablet, laptop), a desktop computer, a server or other computing or processing device for display.

The communication module 352 can be embodied in one or more components that are in communication with each other. The communication module 352 can comprise a wireless transceiver, an antenna, and/or a near field communication (NFC) component, for example, an NFC transponder.

With continued reference to the example implementation of FIG. 3, one or more physiological sensors may gather physiological data from one or more event participants. The one or more physiological sensors can include a variety of different sensors configured to gather various physiologically data. The physiological sensors can include any of the example sensors described herein such as with reference to FIGS. 1A-1F. The physiological sensors may gather physiological data of event participants before, during or after the event. The sensors may be worn by the participants while participating in the event. The sensors may be worn by the participants continuously or periodically.

The sensors may be configured to receive manually entered input (e.g., in response to a prompt) such as from the event participants. For example, a player may be able to press a button on the sensor indicating their level of pain, fatigue, shortness of breath or the like.

The sensors may be configured to communicate with the control system 350 and may transmit physiological data to the control system 350. For example, the sensors may be in communication with, and transmit data to, the communication module 352 of the control system 350. The sensors may be in continuous or periodic communication with the control system 350. For example, communication may be established between the sensors and control system 350 continuously or at set times or intervals or in response to user input. The sensors may transmit data continuously and in real-time or near real-time, to the control system 350. For example, the sensors may transmit physiological data to the control system 350 as the sensor gathers such physiological data such that the delay between acquiring, processing and/or transmitting such physiological data may be small and imperceptible to human senses. The sensors may transmit data at periodic intervals, or in response to a request or command. The sensors may transmit processed and/or unprocessed physiological data to the control system 350.

With continued reference to the example implementation of FIG. 3, the control system 350 may be in communication (e.g., via the communication module 352) with a database 310. The database 310 may store event related data. Event related data may include any data relating to the event such as a score of the event, a time transpired or remaining during the event, mistakes, errors, fouls, strikes, actions (such as a kill in a video game), achievement, completion of a level or benchmark, satisfied goal, or any other participant action or statistic that may be relevant to the particular event. Event related data can also include information relating to a player, such as participant statistics including participant's previous wins and losses, score totals, performance metrics, and the like. Event related data can also include date, time, weather conditions, such as humidity or temperature, and the like.

Event related data can include data relating to the present event such as date of the present event. Event related data can include real-time data such as a score that represents an actual score of the event in real-time. Event related data can include historical data such as data relating to previous events that have since terminated and/or data relating to an earlier portion of the current event.

The control system 350 may request and/or access the database 310 to retrieve data (e.g., event related data) therefrom. In some embodiments, the database may automatically transmit data to the control system 350 such that the control system 350 receives the data in real-time as the database receives or stores the data which may also be in real-time as the event relating to the data occurs. In some embodiments, event related data can be manually entered into the control system 350, for example by an official of the event, such as by a time keeper or score keep or other statistic keeper. The control system 350 can store event related data, such as received from the database 310, in the storage device 356.

With continued reference to the example implementation of FIG. 3, the control system 350 may be in communication (e.g., via the communication module 352) with one or more display devices 320. The display device 320 may be remote to the control system 350. The control system 350 may communicate with the display device 320 via a wired and/or wireless communication. The control system 350 may communicate with the display device 320 via a computing network 330, as shown. The network 330 may comprise a local area network (LAN), a personal area network (PAN) a metropolitan area network (MAN), a wide area network (WAN) or the like, and may allow geographically dispersed devices, systems, databases, servers and the like to connect (e.g., wirelessly) and to communicate (e.g., transfer data) with each other. The control system 350 can establish connection via the network 330 to the display device 320.

The control system 350 can be configured to transmit data to the display device 320. The control system 350 may transmit data to the display device in real-time as the data is received by the control system 350 from other devices or systems such as from physiological sensors. The control system 350 may transmit data to the display device 320 at a time after it has been received by the control system 350 (e.g., not in real-time). For example, the control system 350 may transmit data to the display device 320 that is stored in the storage device 356, such as historical physiological data and/or historical event related data. In embodiments, including more than one display device, the control system 350 can transmit data to separate display devices that is not the same. For example, the control system 350 may transmit first display data to a first display device and second display data to a second display device. The control system 350 may transmit unique data to separate display devices based on unique requests to display data received from different display devices or users.

The display device 320 includes an interface 322. The interface 322 may comprise a display such as a screen for displaying images, videos, or other graphical representations. The display device 320 may be configured to display (e.g., via the interface 322) one or more images, videos, animations or the like in conjunction with and/or which may relate to, the physiological data or the event.

A viewer may view the display device 320 to view the event or data related thereto such as relating to physiologically data of the event participants. A viewer may view, via the display device 320, the event in real-time with the event. A viewer may view, via the display device 320, physiological data of the event participants in real-time with the physiology of the participants and/or in real-time with the event. A viewer may be anyone interested in the participants' physiological data or the event. For example, a viewer may be a player or other participant in the event, a coach, a fan, a spectator, an official such as a referee, a manager and/or an owner of the event, the team, or a player. Additionally, viewers may include those in attendance at the event, or those who are geographically distant from the event, such as those viewing the event over a network such as internet or cable.

The interface 322 may comprise an interactive graphical user interface which may be configured to receive a user input. The display device 320 may be configured to transmit data to the control system 350. For example, the display device 320 may receive a user input via the interface 322 and may transmit the user input to the control system 350.

In some embodiments, the display device 320 may include a television, a mobile device, a phone such as a smartphone, a laptop, a computer, a tablet, a virtual reality (VR) system or device such as a VR headset, an augmented reality (AR) system or device such as an AR headset, or the like. In some embodiments the display device 320 may be remote to the event such as a television at a geographic location distant to the event. In some embodiments, the display device 320 may be at or near the event such as a screen located above the event and displaying the event in real time which may be viewed by spectators of the event. In some embodiments, the display device 320 may be in possession of or held by a participant, a coach an event official or the like. In some embodiments, the display device 320 may be integrated with the physiological sensors of the event participants or otherwise comprised as part of an integrated unit or device with the sensors. For example, a participant may wear a device on their wrist such as a watch which may include physiological sensors and a display screen.

In some implementations, the control system 350 may transmit data to the display device 320 to provide feedback for adjusting a performance of a participant in the event. For example, a participant may view physiological data via the display device 320 as received from the control system 350, and may adjust their technique, strategy, and/or performance accordingly. A participant's own physiological data displayed via the display device 320 may provide performance feedback to the participant and/or the physiological data of the participant's competitor may provide performance feedback to the participant. In some implementations, the physiological data of another person may provide performance feedback to an event participant. For example, a public speaker may be able to view physiological data of audience members in real-time with their speech and may adjust their speech according to the audience member's physiological data. As another example, a player in a sports competition may view the physiological data of an official of the game (e.g., referee) and adjust their playing techniques accordingly (e.g., to avoid incurring a certain call from the official.

The system 300 may optionally include a broadcast device or system 340. The broadcast device or system 340 may be in communication with the database 310, the control system 350, and the display device 320. The broadcast device or system 340 may broadcast the event. The broadcast device or system 340 may receive data from the control system 350, the database 310, and may package the data for broadcasting with the event. In some embodiments, the broadcast device or system 340 may be a streaming media server.

### Example Controller Implementations

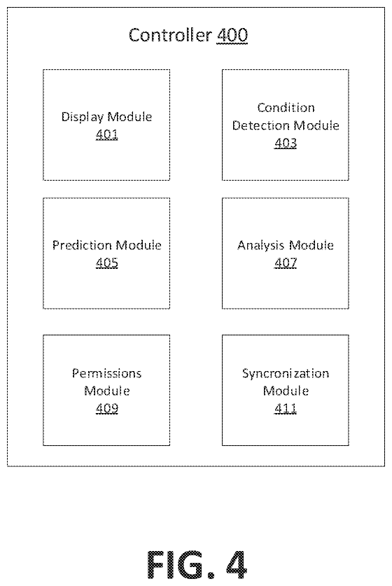

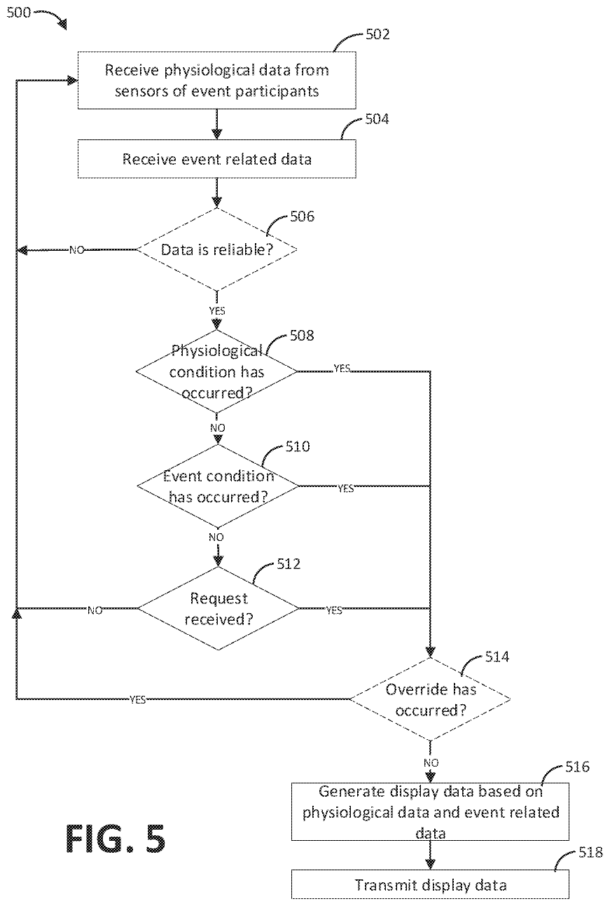

FIG. 4 is a block diagram illustrating an example controller 400 for controlling the display of physiological data and/or event related data. The controller 400 may include software instructions that can be executed (e.g., by a processor) to perform one or more operations or functions. The controller 400 (or modules thereof) can be executed by any variety of computing devices, systems or processors. For example, the controller 400 (or modules thereof) can be executed by the control system 350 (e.g., by processor 354), the physiological sensors, or the display device 320, as shown with reference to FIG. 3. The controller 400 (or modules thereof) may be executed by cloud computing.

As shown in the example embodiment of FIG. 4, the controller 400 includes a display module 401, a condition detection module 403, a prediction module 405, an analysis module 407, a permissions module 409, and a synchronization module 411.

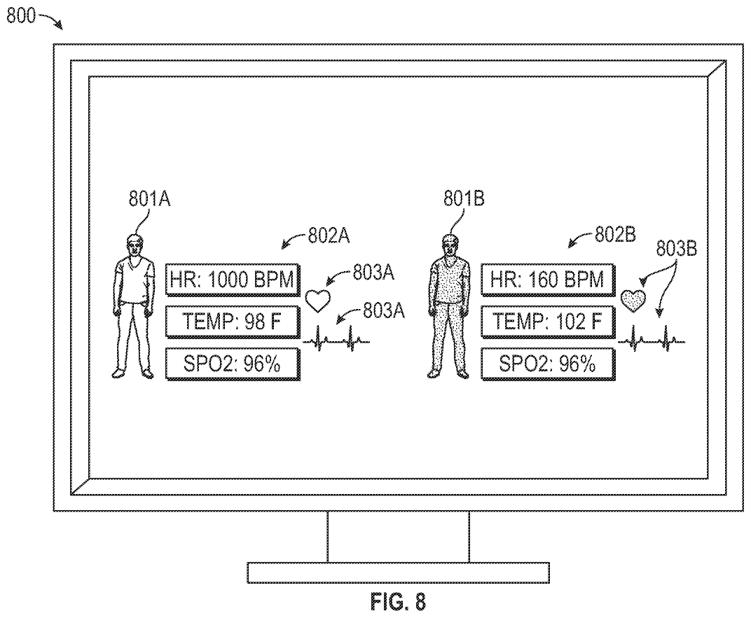

The display module 401 may be configured to generate display data to render a display. For example, the display module 401 may generate display data that is transmitted to a display device (e.g., display device 320) to be rendered by the display device into a visual display. The display module 401 can generate data based on physiological data received from the sensors and/or event related data. The display module 401 can generate data for rendering images, graphics, videos, animations or the like. The display module 401 may be configured to arrange data in various visual formats such numbers, tables, charts, graphs, and the like. For example, the display module 401 may arrange data to be displayed in a bar chart, pie chart, line chart, 3D chart, and the like. The display module 401 may display data for individuals or groups of individuals (e.g., teams). For example, the display module 401 may arrange an individual participant's physiological data in a chart or graph, or may arrange a team's combined or average physiological data into a chart or graph. Example displays which may be rendered and displayed based on data generated by the display module 401 may be shown with reference to FIGS. 8A-8O.

In some embodiments, the display module 401 may generate display data pertaining to one event to be displayed simultaneous with the display of another event. For example, the display module 401 may generate display data for displaying a first event while simultaneously displaying (e.g., in a bottom portion of a display) the physiological data of participants of a second event that is occurring simultaneous to the first event. Thus, viewers may view the first event while also viewing information relating to the second event, when they may not otherwise be able to view both events because they are occurring simultaneously.

In some embodiments, the display module 401, generate display data for superimposing images on physical surface located at the event. For example, the display module 401 use a physical surface at the event, such as a ground surface, as a “green screen” on which to superimpose images (e.g., physiological data) as if the images were actually imprinted, displayed, or otherwise located on the physical surface at the event, from the perspective of the viewer of the display.

In some embodiments, the display module 401 may generate display data to display a replay of preceding events such as a replay of an action that occurred in the event immediately preceding the replay. The display module 401 may combine physiological data to be displayed in the replay whereas such physiological parameter were not displayed during the real-time display of the event.

In some embodiments, the display module 401 may generate display data for displaying images or videos of various products, for example energy drinks or energy bars. The display module 401 may generate display data to display these products in association with (e.g., adjacent to) the physiological data of the players.

With continued reference to FIG. 4, the condition detection module 403 may be configured to detect conditions or events such as physiological conditions, event conditions, or a request or command, which may trigger subsequent actions by the controller 400. For example, the condition detection module 403 may detect that a certain physiological condition has occurred (e.g., participant HR has exceeded a threshold) based on physiological data received from a sensor and, in response, may initiate generation of display data (e.g., by the display module 401). As another example, the condition detection module 403 may detect that a certain event condition has occurred (e.g., an event score has changed, a break or timeout in the event has occurred) based on event related data and, in response, may initiate generation of display data (e.g., by the display module 401). As another example, the condition detection module 403 may detect that a request to display certain information has been received (e.g., from a user via an interactive interface of a display device) and, in response, may initiate generation of display data (e.g., by the display module 401).

With continued reference to FIG. 4, the prediction module 405 may make predictions. The predictions may be based, at least in part, on physiological data (real-time or historical), and/or event related data (real-time or historical). The prediction module 405 may predict a participant's future physiological state. For example, the control system 500 may determine that a participant is likely to experience an average heart rate of 130 beats per minute throughout the duration of the event or that a participant will likely begin to experience fatigue or muscle cramps within a certain time frame. As another example, the prediction module 405 may determine and/or predict a participant's mental and/or emotional state such as, that a participant is experiencing, or is likely to experience, high levels of mental stress, or that a player is nervous or anxious. The prediction module 405 may predict a participant's performance in the event. For example, the prediction module 405 may predict that a participant is likely to score within a certain time interval or will experience impaired performance within a certain time frame. The prediction module 405 may predict an outcome of the event. For example, the prediction module 405 may predict who will win and who will lose an event, the final score of the event, as well as probabilities associated with such predictions.

In some embodiments, the prediction module 405 may be implemented in conjunction with in-game betting. For example, the prediction module 405 may inform in-game betters or gamblers of the probabilities of certain event outcomes (e.g., win/loss probabilities, final score, etc.) which may affect betting and gambling decisions. As another example, the prediction module 405 may inform the payout associated with bets (e.g., based on probabilities of events occurring).

The prediction module 405 may determine appropriate actions and/or suggestions for a participant to take. For example, the prediction module 405 may determine that a participant should drink water, or rest for five minutes. These suggestions may be for actions that can be taken by the participant in real-time, or may be for actions that the participant could have taken prior to the event or could take preceding any future event. For example, the prediction module 405 may determine that a participant should have consumed a particular food prior to the event to increase their blood oxygen saturation (SpO2). As another example, these suggestions may be for actions that a participant may take after the event for example to help with recovery.

In some embodiments, the prediction module 405 may implement one or more machine learning algorithms. Machine learning is a sub-field of computer science based on the study of pattern recognition and computational learning theory in artificial intelligence. It includes the development of algorithms that can learn from and make predictions on data. Algorithms developed through machine learning operate by building a model from example inputs in order to make data-driven predictions or decisions, rather than following strictly static program instructions. Machine learning is employed in a range of computing tasks where use of explicit computer programs is infeasible. When employed in industrial contexts, machine learning methods may be referred to as predictive analytics or predictive modelling. As applied in the present disclosure, the machine learning may include supervised learning, where the machine learning algorithm is presented with training data that include example inputs and their known outputs, given by a “teacher”, and the goal is to learn a general rule that maps the inputs to the outputs. In an embodiment, Fisher's linear discriminant is employed to derive predictions as described herein. Fisher's linear discriminant is a method used to find a linear combination of features that characterizes or separates two or more classes of objects or events. The resulting combination may be used as a linear classifier or for dimensionality reduction before later classification. Other methods of machine learning that can be used with the present disclosure include, without limitation, linear discriminant analysis, analysis of variance, regression analysis, logistic regression, and probit regression, to name a few. A skilled artisan will recognize that many other machine learning algorithms can be used to determine predictions, as discussed herein, without departing from the scope of the present disclosure.

With continued reference to FIG. 4, the analysis module 407 may be configured to perform manipulations and/or calculations on data such as physiological sensor data. For example, the analysis module 407 may process raw or unprocessed physiological data or signals from the physiological sensors and may compute one or more physiological parameters therefrom. In some embodiments, the analysis module 407 may analyze processed physiological data such as physiological parameters or waveforms. For example, the analysis module 407 may calculate averages, minimums, maximums, rates, trends, percentages and the like.

The analysis module 407 may compare event participants according to one or more metrics such as a real-time and/or historical physiological parameter. For example, the analysis module 407 may rank event participants participating in an event by their real-time blood oxygen saturation (SpO2) or other physiological data. As another example, the analysis module 407 may rank players according to an overall real-time physiological state based on one or more physiological parameters which may be gathered by sensors. The analysis module 407 may compare real-time data with historical data. For example, the analysis module 407 may compare a present physiological parameter with a historical physiological parameter from the same event or previous events.

The analysis module 407 can combine physiological data or event related data of one or more participants such as participants on the same team. For example, the analysis module 407 may determine an average physiological parameter for all participants on a team or a portion of participants on a team (e.g., divided by role).

With continued reference to FIG. 4, the permissions module 409 may be configured to determine and implement permissions associated with data such as physiological data. For example, permissions may restrict access to, or prevent data from being transmitted or displayed. As an example, a player, a coach, an owner, broadcast network, a franchise, a sponsor, an agent or some other person or entity with an interest in the data (such as a financial interest, medical interest, privacy interest) may be granted control over the data to implement permissions such that only those who have been granted permission may access and/or view the data. For example, an event participant may grant access to, ownership of, or control over their physiological data to a team owner such as by selling rights to the data. The team owner may likewise grant access, ownership, or control over such physiological data to a broadcast network. The broadcast network may broadcast the event and may only allow viewers of the event to view the participant's physiological data if the viewer has paid or taken some other action to be granted a level of access to the data. For example, a viewer may pay a subscription to view data associated with certain athletes in a sports league or with certain sports teams, or a viewer may download an application for a mobile device to receive access to the data.

Various permission levels may exist which may grant various rights. For example, one permission may allow data to be viewed only in real-time with an event, while another permission may allow data to be downloaded and stored and/or to accessed as historical data, while another permission may allow data to be sold.

With continued reference to FIG. 4, the synchronization module 411 may synchronize (e.g., received data such as physiological data or event related data). For example, the synchronization module 411 may synchronize received physiological data with event related data based on a time at which the data is received and/or a time at which the data is gathered or occurs or events giving rise to the data occur.

The synchronization module 411 may insert tags, time stamps, or markers into the received data to identify a time corresponding with the data to facilitate synchronizing the data with other data. As an example, the synchronization module 411 may insert a tag associated a time X with a data point of physiological data. The synchronization module 411 may also insert a tag associated the time X with a data point of event related data. Because the physiological data and event related data both have the tag identifying time X, the synchronization module 411 can synchronize the data associate with time X from both the phycological data and the event related data with each other.

In some embodiments, the synchronization module 411 may synchronize data (e.g., physiological data with event related data) based on time and/or based on reliability of the data. For example, the synchronization module 411 may synchronize physiological data with event related data if the physiological data has a reliability index or confidence measure above a certain threshold and/or is within a certain time range of the event related data. As one example, the synchronization module 411 may synchronize physiological data with event related data, although they may not occur at the same time, if the synchronized physiological data is the most reliable among a series of physiological data within a timeframe.

### EXAMPLE PROCESSES

FIG. 5 is a flowchart illustrating an example process 500 for generating display data for displaying event related data and/or participant physiological related data. The process 500, or any portion thereof, can be performed on any computing device such as processor 354 described with reference to FIG. 3.

At block 502, a processor can receive physiological data from one or more physiological sensors. The physiological sensor(s) can include various types of sensors and may gather a variety of data. The physiological sensor(s) can be attached to, secured to, worn by, or otherwise connected to one or more event participants. The physiological data may include raw or unprocessed data such as raw signals. The physiological data may include processed data such as physiological parameters, waveforms, indices, or the like. The physiological data may include current or real-time physiological data representing the physiology of the event participant at a time that is substantially the same time as it is received by the processor (e.g., neglecting small time delays which may be imperceptible to human senses). The physiological data may include historical physiological data which may include data that was previously received by the processor (e.g., and stored in a storage device or medium). The historical physiological data may include data that relates to (e.g., was gathered from an event participant during) the same event in which the participant is currently participating, or previous events in which the participant may have previously participated.

At block 504, the processor can receive event related data. Event related data may be received from a database or may be manually inputted to the processor. Event related data may include current or real-time event related data. For example, the event related data may include a score that represent a current actual score of the event. The event related data may include historical event related data which may include data that was previously received by the processor (e.g., and stored in a storage device or medium) and/or data that is received by the processor as historical data. The historical event related data may include data that relates to the same event in which the participant is currently participating, or previous events in which the participant may have previously participated.

At block 506, the processor can optionally determine whether the data is reliable. The data may be physiological data and/or event related data. As an example, the processor may determine that the received physiological data is unreliable if the event participant is undergoing significant amounts of motion (e.g., as determined by a sensor configured to determine motion, orientation, acceleration, etc.). As another example, the processor may determine that the received event related data is not reliable if a time elapsed after the event related data has occurred has not exceeded a threshold. For example, the processor may wait a certain length of time after a score has changed to make sure the score will remain changed (e.g., will not be recalled by the officials etc.). As another example, the processor may determine that the received event related data is not reliable if it conflicts with other event related data or depends on other event related data. For example, event data such as a change in score or a foul, may not be reliable if it is being challenged by a player or coach and is currently under review by officials for validation or recall. The processor may not generate display data if the received physiological and/or event related data is unreliable.

At block 508, the processor can detect whether a physiological condition has occurred. The physiological condition can include a variety of conditions, states, criteria, of any number of participants. Physiological conditions can include, for example, physiological parameters, such as HR, RR, temperature, SpO2, exceeding a certain threshold. Physiological conditions can include combinations of conditions. For example, a condition may be determined to have occurred if a participant's heart rate exceeds a threshold for a certain period of time and the participant's temperature is above a threshold level. Physiological conditions can include comparisons between participants. For example, a condition may be determined to have occurred if a certain physiological parameter of one participant differs from that of another participant by a certain margin. The occurrence and detection of a physiological condition may trigger the processor to generate display data at block 516.

At block 510, the processor can detect whether an event condition has occurred. Event conditions can include a variety of conditions or combinations of conditions which may relate to the event. Example event conditions can include a score exceeding a certain threshold, the difference in scores between participants exceeding or within a threshold, a change in score, occurrence of a timeout or break or halftime, a certain time remaining in the event, occurrence of an error or mistake, completion of an action by a participant such as scoring a point, participant breaking a record, participant exceeding past performances in previous events or the same event, a velocity exceeding a threshold (such as during a race), commencement of the event, termination of the event, physical injury or extreme exertion, and the like. The occurrence and detection of an event condition may trigger the processor to generate display data at block 516.

At block 512, the processor may determine whether a request has been received. The request may be a request to display data (e.g., physiological or event related data). The request may be received from a user or viewer via computing device such as a computing device configured to display the event or related physiological data.

At block 514, the processor may optionally determine whether an override has occurred. An override may prevent the processor from generating and/or transmitting display data. An override may be generated by anyone with an interest in the data (e.g., physiological data). For example, an event participant may choose to prevent their physiological data from being displayed. In some embodiments, a coach, or sports agent, or medical professional may implement an override to prevent data of a participant to be displayed. Overrides can be implemented during an entirety of an event or for portions thereof. For example, a participant can toggle an override as desired during an event. As an example, an athlete competing in a sports competition may allow their data to be displayed while they are playing but may implement an override to prevent their data from being displayed while they are not playing such as when resting during a break or when sitting on the bench while others are playing. As another example, an athlete competing in a sports competition may allow their data to be displayed but if the athlete is injured, a medical professional may implement an override to prevent the player's data from being displayed such as when medical care is being provided to the player.

In some embodiments, an override may be implemented by a broadcast network, sports club, franchise, sponsor, or other entity such as with a financial interest in the event. For example, a network that broadcasts sports events may implement a default override unless a viewer has a paid subscription for viewing physiological data of sports players. Such an override (or subscription) may be per game, per team, per player, per time, or the like. As another example, an owner of a sports team may implement a default override for the team or players thereof unless a broadcast network has paid the owner to be able to display physiological data of the team members.

In some embodiments, multiple persons or entities may be able to implement overrides. Any sequence of logic may be implemented when handling multiple overrides. For example, an override may be implemented if any of multiple persons or entities action an override or an override will by only be implemented if multiple certain persons or entities action their respective overrides.

At block 516, the processor may generate display data. The processor may generate display data in response to any of blocks 508, 510, 512 occurring individually or in combination. For example, in some embodiments, the processor may only generate display data at block 516 if both a physiological condition has been detected at block 508 and an event condition has been detected at block 510. The display data may be used to render a graphic, image, video, animation or the like on a display device such as a display screen. The display data may render images etc. of, relating to, or representing, physiological data and/or event related data. For example, the display data may render representations of physiological parameters in combination with participant statistics. In some embodiments, the processor may generate audio data in combination with the display data. For example, the processor may generate one or more sounds to be outputted in combination with the display data. audio data may complement or supplement or correspond with any visual images of the generated display data. As an example, the processor may generate the sound of a beating heart to be outputted simultaneous to the display of a beating heart icon or the display of an ECG waveform.

The processor may generate display data based on the received physiological data and/or the received event related data. For example, the processor may generate display data for rendering a display including an animation of a participant with a certain color depending on a physiological status of the participant (e.g., red if temperature exceeds threshold, blue if SpO2 exceeds threshold). As another example, the processor may generate display data to render an image etc. in an entirety of a display screen if a break in the event has occurred (e.g., a timeout), or to render an image in only a portion of a display screen if the event is ongoing to allow a viewer to view the event and the rendered image simultaneously.

In some embodiments, the display data generated at block 516 may include portions of the event that have already occurred. For example, the generated display data may include a replay of a portion of the event. As an example, the processor may combine physiological data with segments of an event to be replayed so that a viewer may rewatch an interesting portion of the event (e.g., immediately after) with extra information (e.g., physiological data).

At block 518, the processor may transmit the display data, for example to one or more display devices, which may render and display the display data.

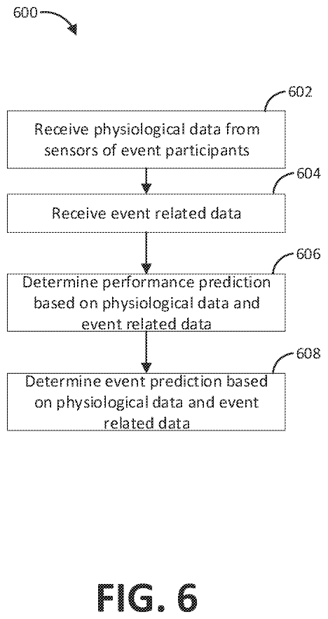

FIG. 6 is a flowchart illustrating an example process 600 for predicting participant performance or event outcome. The process 600, or any portion thereof, can be performed on any computing device such as processor 354 described with reference to FIG. 3.

At block 602, a processor can receive physiological data from sensors of event participants. At block 604, the processor can receive event related data. The physiological data and event related data can include present or real-time data and/or historical data, for example, as discussed with reference to blocks 502 and 504 of FIG. 5.

At block 606, the processor may determine a performance prediction. The performance prediction may include a prediction about a participant's performance or about a team's performance. For example, the processor may predict that a participant is likely going to score a point within a certain time frame. As another example, prior to commencement of the event, the processor may determine that a participant will score a certain number of total points during the event. The performance prediction may be based on the received physiological data and/or event related data. For example, the processor may compare a current physiological state (e.g., based on current physiological data) with a previous physiological state (e.g., based on historical physiological data) as well as previous event related data to predict performance during the present event.

At block 608, the processor may determine an event prediction. The event prediction may include a prediction about an outcome of the event, such as who will win, who will lose, rank of participants from winner to loser, a final score, the probabilities associated with such predictions, and the like. In some embodiments, such event predictions may inform in-game betting decisions. As discussed above with reference to block 606, the event prediction may be based on the received physiological data and/or event related data.

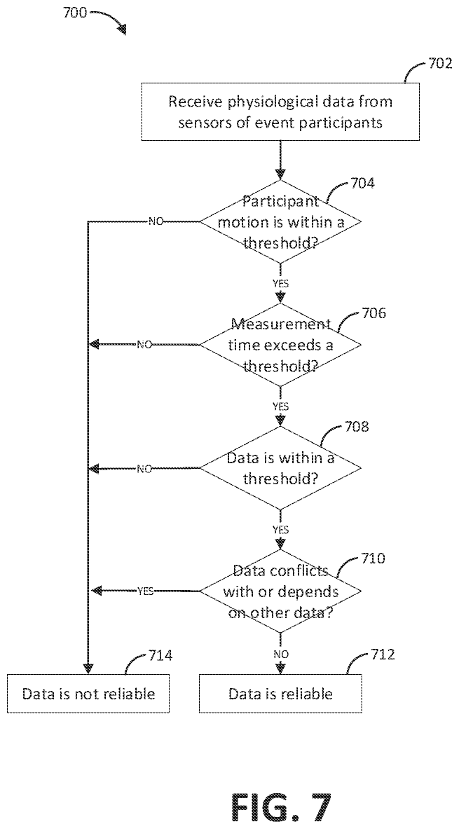

FIG. 7 is a flowchart illustrating an example process 700 for determining the reliability of data. The process 700, or any portion thereof, can be performed on any computing device such as processor 354 described with reference to FIG. 3.

At block 702, the processor can receive physiological data from sensors of event participants. The physiological data can include present or real-time data and/or historical data, for example, as discussed with reference to blocks 502 and 504 of FIG. 5.

At block 704, the processor can determine whether a participant's motion is within a certain threshold. Participant's motion can include orientation, position, acceleration and the like, and may be determined by one or more sensors attached to, worn by, or otherwise connected to the participant, such as accelerometers, gyroscopes and the like. In some embodiments, a sensor, such as the sensor shown in FIG. 2E, may be configured to gather physiological data and motion-related data. If, at block 704, the processor determines that the participant's motion exceeds a threshold, the processor may determine that some or all of the physiological data gathered from some or all of any of the sensors attached to the participant may not be reliable. For example, motion may introduce noise into the sensor signals which may reduce a quality of the resulting data.

At block 706, the processor can determine whether a time that a sensor has been measuring or collecting data exceeds a certain threshold. The time threshold may be unique for any of the various sensors attached the participant. Physiological sensors attached to a participant may need a certain time to calibrate after being turned on or after commencing measurements before resulting data is sufficiently reliable. If, at block 706, the processor determines that the time does not exceed a threshold, the processor may determine that some or all of the physiological data gathered from some or all of any of the sensors attached to the participant may not be reliable.

At block 708, the processor can determine whether the physiological data from the sensors is within a certain threshold. For example, the processor may determine whether the data includes any outliers, exceeds a predefined physiological reality, or the like. If, at block 708, the processor determines that the data exceeds a threshold, the processor may determine that some or all of the physiological data gathered from some or all of any of the sensors attached to the participant may not be reliable.

At block 710, the processor can determine whether received physiological data conflicts with or depends on other data, including other physiological data or event related data. For example, the processor may determine whether related data from multiple sensors is consistent or inconsistent. As an example, a first and second sensor attached to the same participant may both measure the participant's blood oxygen saturation. The processor can determine if the measurements from these first and second sensors conflicts with each other. If, at block 710, the processor determines that the data conflicts with or depends on other data, the processor may determine that some or all of the physiological data gathered from some or all of any of the sensors attached to the participant may not be reliable.

At block 712, the processor can output a determination that the data is reliable. At block 714, the processor can output a determination that the data is not reliable. In some embodiments, the processor can output a reliability index or score at either of blocks 712 or 714 in addition to, or in place of, the output that the data is reliable or not. The reliability index or score may be based on any of the determinations of blocks 704-710. The reliability index or score may be a measure or confidence of the reliability of the data. In some embodiments, the processor can filter data that is not reliable or which has a reliability index or score below a threshold level. For example, the processor may discard or reject such data. In some embodiments, the processor can assign a reliability index or score of zero to data to indicate that the data should not be considered.

FIG. 7 is shown as an example is not intended to be limiting. In some embodiments, the process 700 may include less blocks than those shown. For example, the process 700 may only include block 704. In some embodiments, the process 700 may include more blocks than those shown. For example, the process 700 may only include additional blocks relating to different metrics for determining data reliability.

### Example Display Embodiments

FIGS. 8-23 illustrate example displays for displaying event related data, participant physiology related data, and the like. FIGS. 8-23 are provided as examples and are not intended to be limiting. The features shown in any of FIGS. 8-23 can be reordered, removed, rearranged, or recombined within each of respective FIGS. 8-23 or between any of FIGS. 8-23.

FIG. 8 illustrates an example display 800 for displaying event related data, participant physiology related data, and the like. The display is rendered or displayed in a display device which may be a computer, television, or the like, and to which data has been transmitted.

In the example of FIG. 8A, the display 800 displays a first avatar 801A of a first participant and a second avatar 801B of a second participant. The avatars 801 of participants may be animated representations (e.g., illustrated or computer-generated images) of the participants or photographs of the participants. The avatars 801 may resemble the participants and be recognizable as representing the participants. The avatars 801 may be of the entire body of the participant or a portion of the body of the participant, such as the participant's face. The avatars 801 may be static images or may include video motion. For example, the avatars 801 may move. The avatars 801 may include coloring, shading, etc. representing a physiological state of the participant. For example, as shown, avatar 801A is a first color or shading (such as blue) corresponding to a low body temperature or low heart rate or the like. Avatar 801B is a second color or shading (such as red) corresponding to a high body temperature or high heart rate or the like.

The display 800 displays first physiological data 802A (e.g., physiological parameters) corresponding to physiological data gathered from sensors attached to the first participant. The display 800 displays second physiological data 802B (e.g., physiological parameters) corresponding to physiological data gathered from sensors attached to the second participant. The physiological data 802 includes HR, body temperature, blood oxygen saturation (SpO2). In some embodiments, the physiological data 802 can include more or fewer parameters than shown.

The display 800 displays first animations 803A and second animations 803B which correspond to physiological parameters of the first and second participants, respectively. The animations 803 may include coloring or shading which may correspond to and represent the physiological data. For example, the first animations 803 includes a heart which has a first color or shading (e.g., blue) corresponding to a low heart rate, or body temperature, or SpO2 etc. The second animations 803 includes a heart which has a second color or shading (e.g., red) corresponding to a high heart rate, or body temperature, or SpO2 etc. The first and second animations 803 also include an ECG waveform which correspond to cardiac activity of the first and second participants, respectively. For example, the ECG waveforms may be a real-time ECG waveform displaying real-time cardiac activity of the participants. The first and second animations 803 may be static images or may include video motion. For example, the hearts of animations 803 may beat or pulse at a rate corresponding to a real-time heart rate of the participants. As another example, the hearts of animations 803 may beat with an associated heart beat sound.

The display 800 displays the avatars 801, physiological data 802 etc. in a portion of the display screen which may be less than an entirety of the display screen. Advantageously, this may allow the display screen to simultaneously display other graphics, images, videos, or the like, such as the event, to allow a viewer to view both simultaneously. In some embodiments, the display 800 may display the avatar, physiological data 802 etc. in an entirety of the screen. In some embodiments, the display 800 may display the avatars 801, physiological data 802 etc. in other portions of the display screen such as a central portion or a top portion.

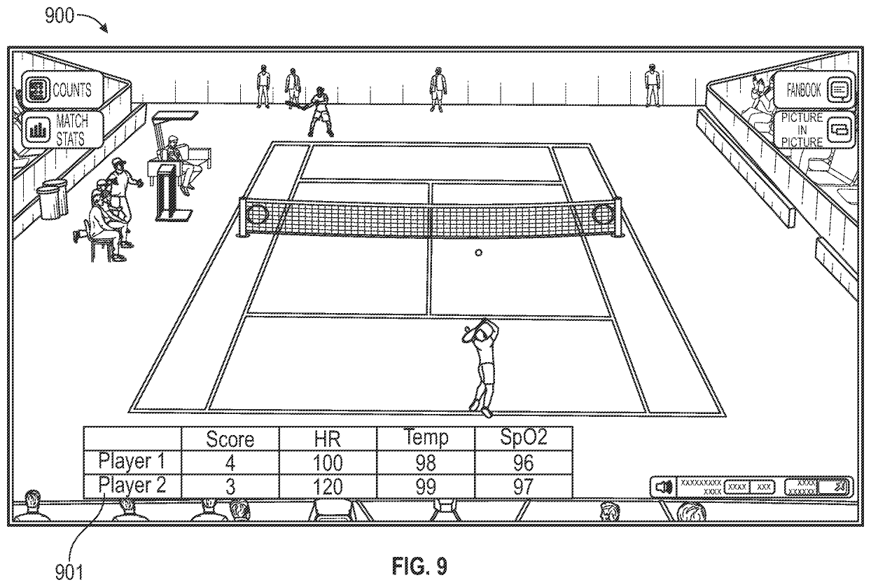

FIG. 9 illustrates an example display 900 for displaying event related data, participant physiology related data, and the like. The display 900 displays a table 901 in a first portion of the display screen and simultaneously displays the event in other portions of the display screen. As shown, the event is a tennis match.

The table 901 includes multiple rows each row corresponding to one of multiple players in the tennis match, such as player 1 and player 2. In some embodiments, the table 901 can include more or fewer rows corresponding to more or fewer players. The table 901 includes multiple columns each column corresponding to one of multiple physiological parameters, such as heart rate, temperature, and SpO2. In some embodiments, the table 901 may display values representing real-time physiological parameters. In some embodiments, the table 901 may display values representing average physiological parameters over a period of time. In some embodiments, the table 901 can include more columns corresponding to more physiological parameters or fewer columns corresponding to fewer physiological parameters. Table 901 also includes a column corresponding to score. In some embodiments, the table 901 can include more columns corresponding to other event related data such as time remaining, fouls, etc. In some embodiments, the table 901 may not include columns corresponding to event related data.

In some embodiments, the table 901 may be updated in real-time with the players' physiology. In some embodiments, the table 901 may be updated periodically such as at fixed time intervals. In some embodiments, the table 901 may be updated in response to the occurrence of a physiological condition such as a player physiological parameter exceeding a threshold. In some embodiments, the table 901 may be updated in response to the occurrence of an event condition such as a change in score, occurrence of a timeout, etc.

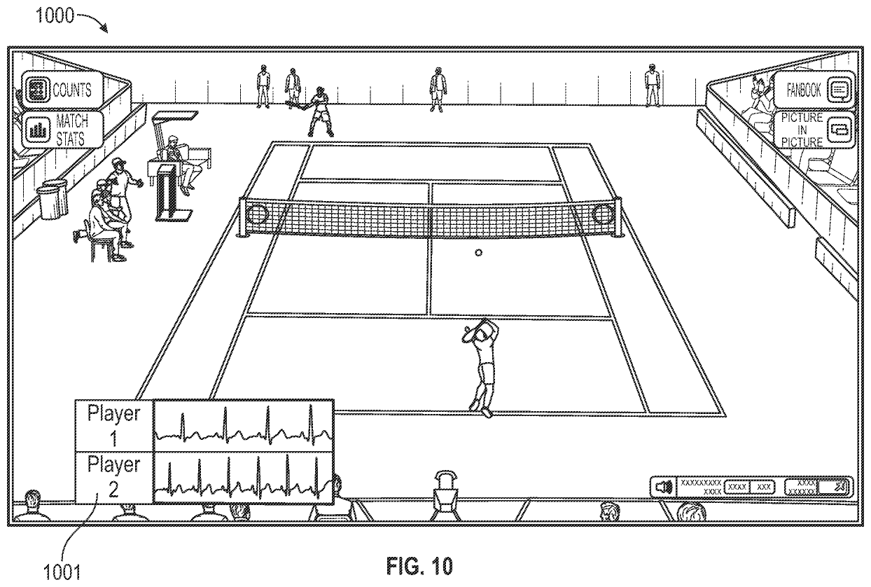

FIG. 10 illustrates an example display 1000 for displaying event related data, participant physiology related data, and the like. The display 1000 displays a chart 1001 which includes rows correspond to various players and columns corresponding to cardiac related physiological data of the players (e.g., ECG waveforms). The ECG waveforms of the chart 1001 may be represent real-time cardiac activity of the players. For example, the ECG waveforms may be displayed in real-time as physiological data is received from physiological sensors and/or in real-time as cardiac activity of the participants occurs. In some embodiments, the ECG waveforms may not correspond directly to cardiac activity of the participants (e.g., may not be an actual ECG waveform of cardiac electrical signals) but rather may generally represent or symbolize cardiac activity. For example, the ECG waveforms may be generic illustrations of ECG waveforms that may include waves or pulse that closer together or farther apart to represent a faster or slower heart rate of the participant.

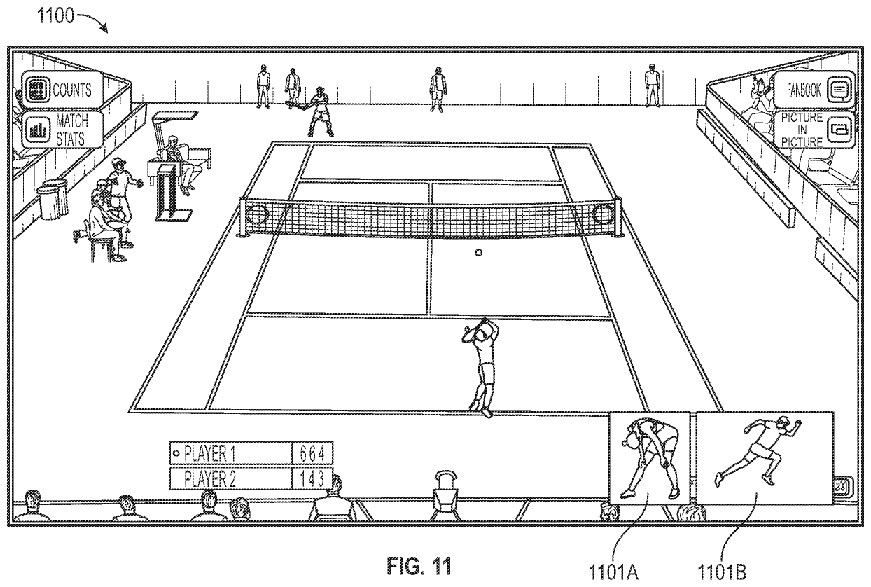

FIG. 11 illustrates an example display 1100 for displaying event related data, participant physiology related data, and the like. The display 1100 displays avatars 1101A and 1101B which correspond to participants in the event. For example, the avatar 1101A may represent and correspond to a first player and the avatar 1101B may represent and correspond to a second player. The avatars 1101 may be videos, video animations, static photographs or illustrations of the participants. The avatars 1101 may move or undergo a sequence of action that corresponds to a physiological state of the participant they represent. For example, as shown, avatar 1101A is shown as bending over with their hands on their knees to represent a certain physiological state of a first participant as determined by their corresponding physiological data (e.g., that the first participant is tired and/or that one or more of the physiological parameters of the first participant have exceeded (above or below) a threshold). As another example, as shown, avatar 1101B is shown in an active state (e.g., running) to represent a certain physiological state of the second participant as determined by their corresponding physiological data (e.g., that the second participant has energy, is performing well, and/or that one or more of the physiological parameters of the second participant have exceeded (above or below) a threshold).

The avatars 1101 may undergo other animated sequences as desired. For example, the avatars 1101 may fall to the ground when the player has a low heart rate. As another example, the avatars 1101 may light on fire when the player's body temperature reaches a certain level or when the player is performing well as determined by physiological data and/or event related data.

In some embodiments, the avatars 1101 may include facial expressions representing a physiological state of the participants as determined by their corresponding physiological data (e.g., positive or negative facial expressions when the player has a high or low blood oxygen saturation (SpO2)). Other similar images may be shown which may assist a viewer in understanding the physiological data or may provide entertainment value to a viewer of the participants' physiological state or data.

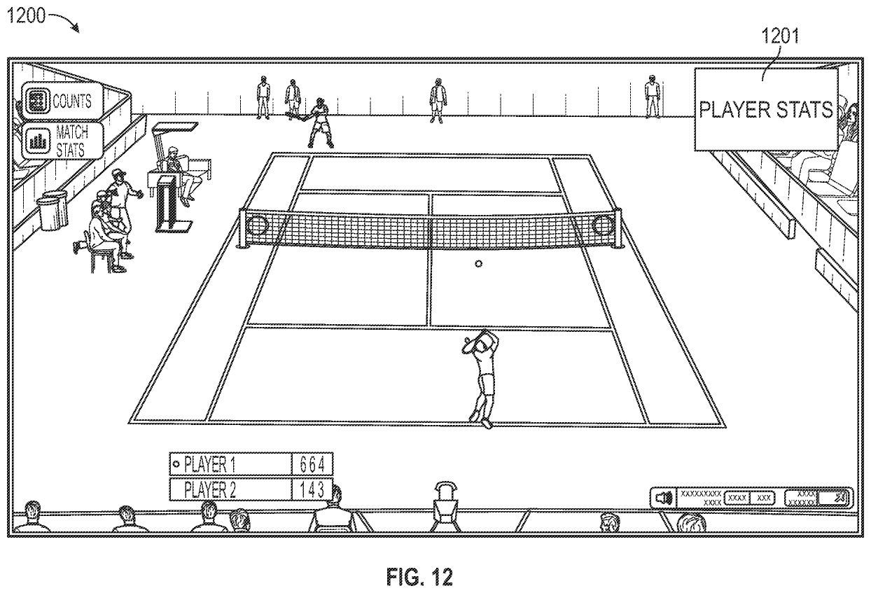

FIG. 12 illustrates an example display 1200 for displaying event related data, participant physiology related data, and the like. The display 1200 includes a selectable component 1201. A user or viewer may select the selectable component 1201. Selecting the selectable component 1201 may cause the display device on which the display 1200 is displayed to transmit data to a remote system (e.g., control system 350 shown in FIG. 3). For example, selecting the selectable component 1201 may cause the display device to transmit a request to a remote system to transmit additional data to the display device to be displayed (e.g., as shown in FIG. 8F).

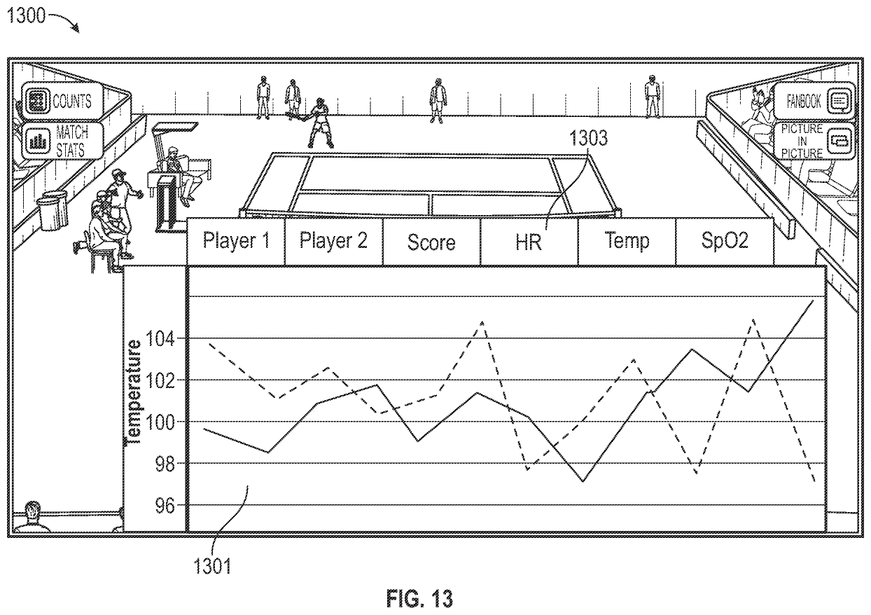

FIG. 13 illustrates an example display 1300 for displaying event related data, participant physiology related data, and the like. The display 1300 displays a graph 1301. The graph 1301 may be displayed in response to a user request (e.g., via the display such as by selecting a selectable component on the display). The graph 1301 includes a top portion including selectable tabs 1303. The selectable tabs 1303 correspond to various metrics which may displayed on the graph 1303. AS shown, the selectable tabs 1303 correspond to one or more participants (e.g., player 1, player 2) and one or more physiological parameters (e.g., HR, temperature, SpO2) and an event related data (e.g., score). In some embodiments, the selectable tabs 1303 may correspond to other metrics such as other participants, other physiological parameters or other event related data.

The selectable tabs 1303 may be selected by a user or viewer which may cause the display 1300 to update the graph 1301 according to which selectable tabs 1303 were selected. In the example of FIG. 8F, a user or viewer has selected “Player 1”, Player 2”, and “Temp” and has not selected the other selectable tabs. Accordingly, the graph 1301 displays a graph of temperature data relating to a first player and a second player for a period of time. In some embodiments, a user or viewer may select as many physiological parameters, event related data, and/or players to combine on the graph 1301.

In some embodiments, a user may select to view data of different participants on the same graph such as is shown. In some embodiments, a user may select to view data from the same participant from different events in which the user has participated. For example, a user may select to view, and the graph 1301 may display a line graph including data of a participant's temperature during a current event, data of the participant's temperature during a first previous event and data of the participant's temperature during a second previous event.

The graph 1301 includes an axis corresponding to time. In some embodiments, the time dimension of the graph 1301 may correspond to a length of time transpired during the event, or may also include previous events.

In some embodiments, a user or viewer may select a type of visualization by which to view the selected data. For example, a user or viewer may select to view the data a line graph such as is shown, or as a bar chart, pie chart, scatter plot, 3D graph, table or the like. In some embodiments, a user or viewer may interact with or manipulate the displayed data. For example, a viewer may be able to calculate averages, minimums, maximums, ranges and the like of the data or may be able to select the graph to view a number value of the data at the selected point of the graph.

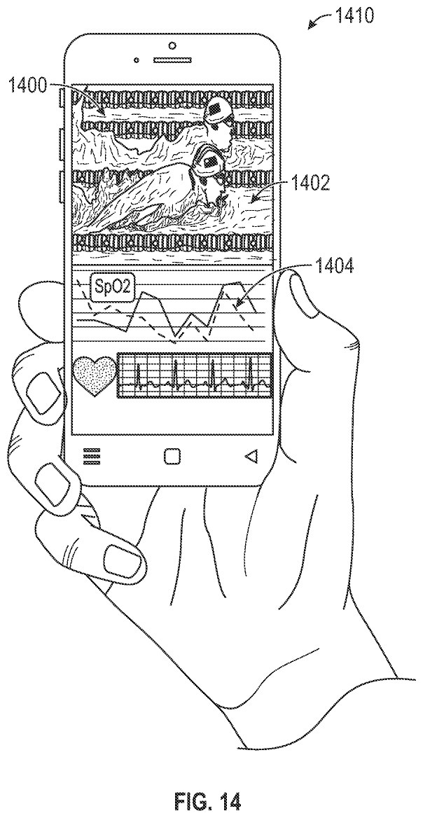

FIG. 14 illustrates an example display 1400 for displaying event related data, participant physiology related data, and the like. The display 1400 is displayed in a display device 1410. The display device 1410 is a mobile device such as a smartphone. The display 1400 includes a first portion 1402 for displaying the event. The event is a swimming event, such as a swimming race. The display 1400 includes a second portion 1404 for displaying physiological related data of the event participants. As shown, the second portion 1404 includes SpO2 trends as well as an ECG waveform.

In some embodiments, the display 1400 may be displayed via a mobile application that may be downloaded or installed on the display device 1410. The mobile application may include instructions (e.g., software instructions) for rendering the display 1400 according to settings of the mobile applications which may be predefined or set by a user.

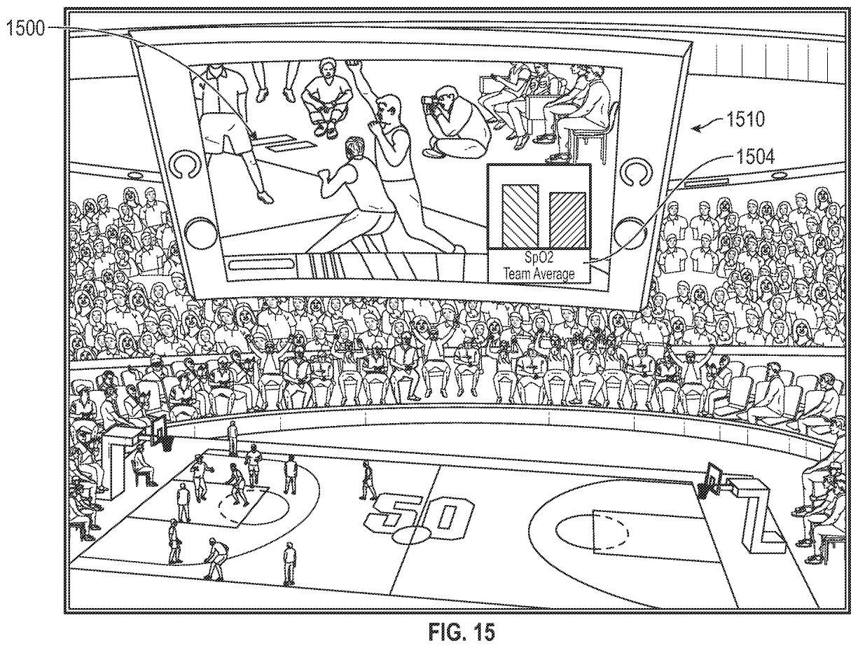

FIG. 15 illustrates an example display 1500 for displaying event related data, participant physiology related data, and the like. The display 1500 is displayed in a display device 1510. The display device 1510 is a large screen, such as a jumbotron, located at a same physical location as the event. In this example, the display device 1510 is located above a basketball game. The display 1500 can be configured to display the event, such as real-time video footage of the event, instant replays and the like. The display 1500 can be configured to display physiological related data of the participants of the event. Display 1500 includes a chart (e.g., a bar chart) 1504 comparing an average SpO2 level of the players of a first team with an average SpO2 level of the players of a second team.

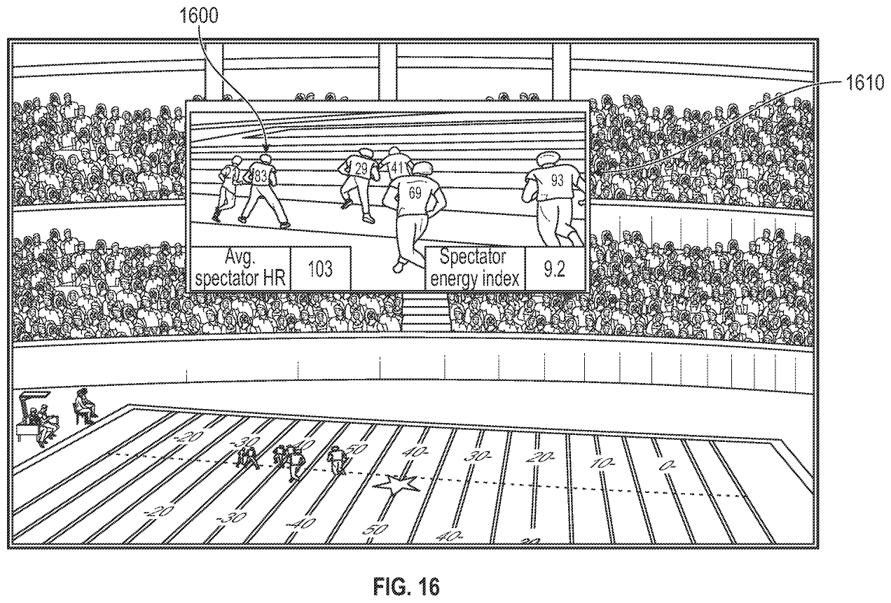

FIG. 16 illustrates an example display 1600 for displaying event related data, participant physiology related data, and the like. The display 1600 is displayed in a display device 1610. The display device 1610 is a large screen, such as a large screen television, located at a same physical location as the event. In this example, the display device 1610 is located above a football game. The display 1600 can be configured to display the event, such as real-time video footage of the event, instant replays and the like. The display 1600 can be configured to display physiological related data. In this example, display 1600 displays physiological related data of the spectators of the event, such as those who are in attendance at the event and who are viewing the event. Display 1600 displays an average spectator heart rate as 103 (e.g., beats per minute) as well as a spectator energy index as 9.2. The spectator energy index may be based on one or more physiological parameters or data. the spectator energy index may be a number between zero and ten to indicate an energy, enthusiasm, or excitement of the spectators, or their level of interest in the game. As an example, some or all of the spectators at the event may wear physiological sensors which may gather and transmit their physiological data to a control system for analysis and display. As another example, any number of spectators, at the event or remote to the event, may optionally gather and transmit their own physiological data (e.g., by using an application of a mobile device such as a smartphone or smartwatch) to a control system for analysis and display.

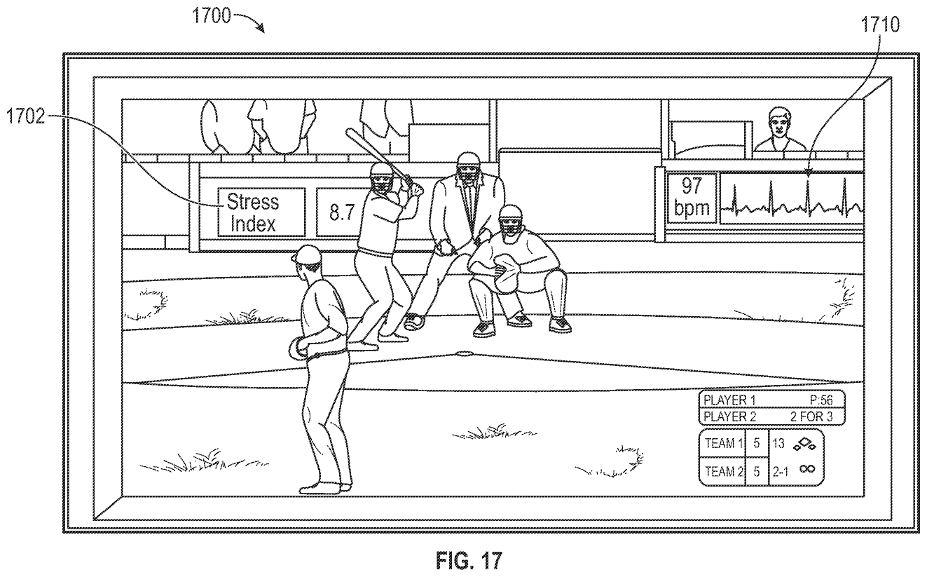

FIG. 17 illustrates an example display 1700 for displaying event related data, participant physiology related data, and the like. The display 1700 may display the event to a viewer of the event who may be remote to the event. In this example, the event is a baseball game. The display 1700 displays a stress index 1702. The stress index 1702 may be based on physiological data gathered from sensors attached to a participant in the baseball game, such as the pitcher, the batter, the catcher, or the umpire. The display 1700 can display the stress index 1702 or other physiological related data as superimposed on a surface at the actual event. For example, the display data used to render the display 1700 may employ “green screen” techniques. Thus, a viewer, viewing the display 1700 may perceive the stress index 1702 as if it were actually imprinted on the surface at the event, whereas a person physically present at the event would not see the stress index 1702 at all. As shown in this example, the stress index 1702 is superimposed on a surface behind the batter such that a portion of the batter's body obstructs a portion of the displayed stress index 1702 from the view of a viewer of the display 1700.

As shown, the display 1700 may display a view of display device 1710 which is physically present at the event. The display device 1710 can display physiologically related data to those who are physically present at the event as well as to those who are remote to the event. In this example, the display device 1710 displays cardiac related activity including an ECG waveform and heart rate. The cardiac activity may be gathered from sensors attached to a participant in the baseball game, such as the pitcher, the batter, the catcher, or the umpire.

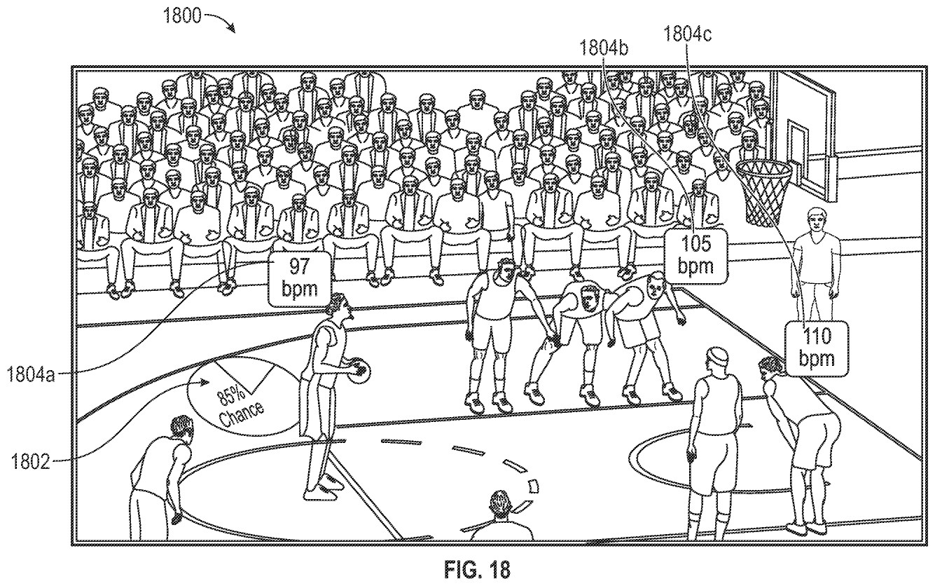

FIG. 18 illustrates an example display 1800 for displaying event related data, participant physiology related data, and the like. The display 1800 may display the event to a viewer of the event who may be remote to the event. In this example, the event is a basketball game. The display 1800 displays a prediction indicator 1802. In this example, the prediction indicator 1802 indicates a likelihood that the player shooting the basketball will successfully complete the shot and score a point. The prediction may be based on physiological data (e.g., historical and/or real-time) associated with the shooting player as well as event related data (e.g., historical and/or real-time). The display 1800 can display the prediction indicator 1802 or other physiological related data as superimposed on a surface at the actual event, such as the floor. For example, the display data used to render the display 1800 may employ “green screen” techniques. Thus, a viewer, viewing the display 1800 may perceive the prediction indicator 1802 as if it were actually imprinted on the floor surface at the event, whereas a person physically present at the event would not see the prediction indicator 1802 at all. As shown in this example, the prediction indicator 1802 is superimposed on a ground surface such that a portion of the shooting player's body obstructs a portion of the displayed prediction indicator 1802 from the view of a viewer of the display 1800.

The display 1800 can display other physiological related data. As shown, the display 1800 displays the heart rates 1804 of certain players. The heart rates 1804 can be displayed adjacent to the player with whom they are associated. The heart rates 1804 can move in the display 1800 as the players move. In some embodiments, the heart rates 1804 may be displayed for players that have selected by a viewer. In some embodiments, the heart rates 1804 may be displayed for players that have unusual heart rates (e.g., unusually high or low). In some embodiments, the heart rates 1804 may be displayed for players that are performing a special action, such as shooting as shot, or undergoing unique circumstances, such as experiencing an injury. In some embodiments, the heart rates 1804 may be displayed at critical, unique, or interesting times during the event, such as when a certain time remains in the event, when a score changes, when a score is close (e.g., tied), and the like.

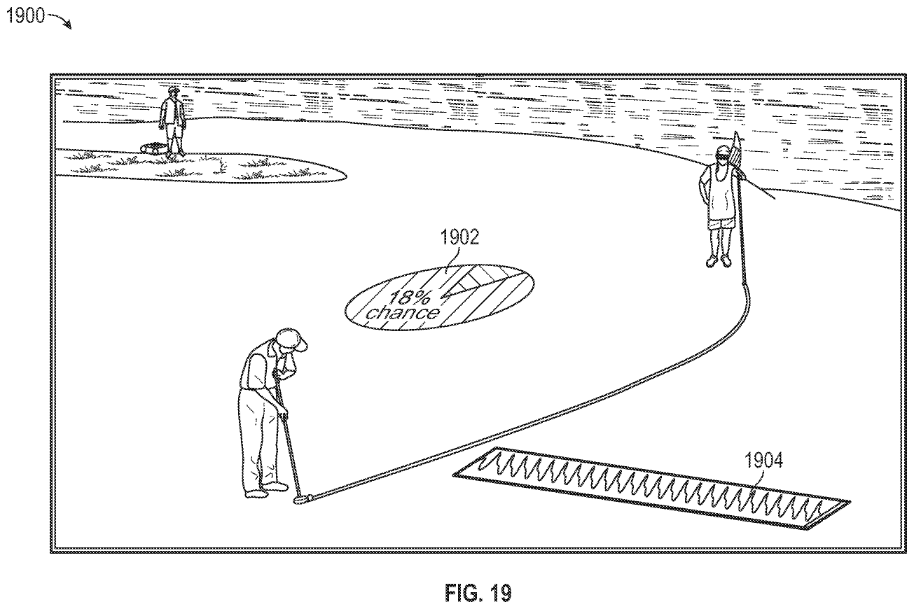

FIG. 19 illustrates an example display 1900 for displaying event related data, participant physiology related data, and the like. The display 1900 may display the event to a viewer of the event who may be remote to the event. In this example, the event is a golf event. The display 1900 displays a prediction indicator 1902. In this example, the prediction indicator 1902 indicates a likelihood that the golfer hitting the ball will successfully hit the ball into the hole. The prediction may be based on physiological data (e.g., historical and/or real-time) associated with the golf player as well as event related data (e.g., historical and/or real-time). The display 1900 can display the prediction indicator 1902 or other physiological related data as superimposed on a surface at the actual event, such as the ground surface. For example, the display data used to render the display 1900 may employ “green screen” techniques. Thus, a viewer, viewing the display 1900 may perceive the prediction indicator 1902 as if it were actually imprinted on the ground surface at the event, whereas a person physically present at the event would not see the prediction indicator 1902 at all.

The display 1900 can display other physiological related data. As shown, the display 1900 displays a plethysmograph waveform 1904. The plethysmograph waveform 1904 may be based on physiological data gathered from sensors of a golfer at the event. In some embodiments, the plethysmograph waveform 1904 may be displayed for players that are performing a special action, such as hitting the ball. In some embodiments, the plethysmograph waveform 1904 may be displayed at critical, unique, or interesting times during the event, such as when a score changes, when a score is close (e.g., tied), and the like.

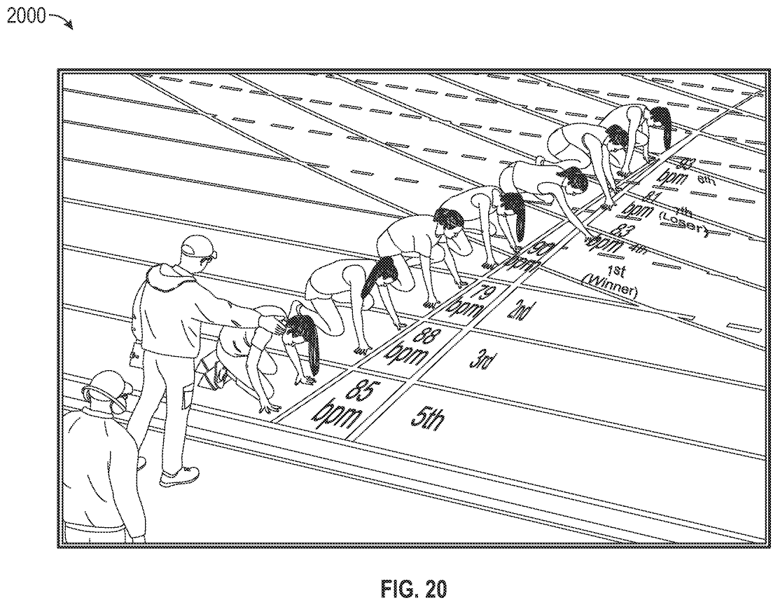

FIG. 20 illustrates an example display 2000 for displaying event related data, participant physiology related data, and the like. The display 2000 may display the event to a viewer of the event who may be remote to the event. In this example, the event is a running event, such as a track and field race. The display 2000 displays data as superimposed on a surface at the actual event, such as the ground surface. For example, the display data used to render the display 1900 may employ “green screen” techniques. Thus, a viewer, viewing the display 1900 may perceive the displayed data as if it were actually imprinted on the ground surface at the event, whereas a person physically present at the event would not see the data at all. In this example, the displayed data indicates a predicted estimated ranking of the runners or order in which the runners will finish the race (e.g., first to last). The prediction may be based on physiological data (e.g., historical and/or real-time) associated with the runners as well as event related data (e.g., historical and/or real-time). In this example, the displayed data also includes heart rates of the runners.

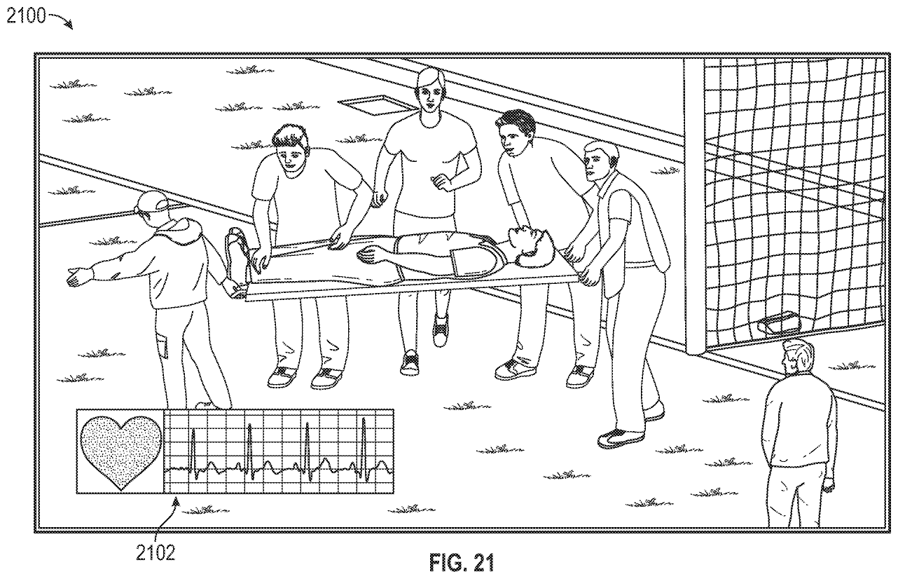

FIG. 21 illustrates an example display 2100 for displaying event related data, participant physiology related data, and the like. The display 2100 may display the event to a viewer of the event who may be remote to the event. In this example, the event is a soccer game. The display 2100 includes cardiac related data 2102 including an ECG waveform and a heart icon. The heart icon may change color, shape, or size depending on related physiological data to indicate a physiological (e.g., cardiac) status of a participant. The cardiac related data may be derived from physiological data of sensors attached to the injured player. The display 2100 may selectively display the cardiac related data 2102, or other physiological related data, at critical, unique, or interesting times during the event such as during a medical timeout, as shown. The display 2100 may display the cardiac related data 2102, or other physiological related data, at times during the soccer game when the players are not playing, as shown, or when the players are playing.

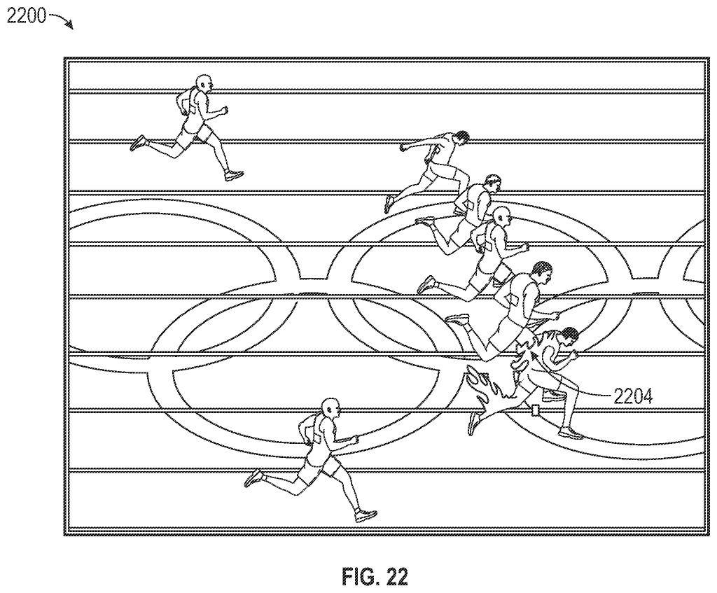

FIG. 22 illustrates an example display 2200 for displaying event related data, participant physiology related data, and the like. The display 2200 may display the event to a viewer of the event who may be remote to the event. In this example, the event is a running event, such as a track and field race. The display 2200 includes animations 2204. The animations 2204 are associated with a runner. The animations are fire or flames that may appear, to a viewer of the display 2200, to be emanating from the runner. The animations 2204 may provide entertainment value to a viewer of the event. The animations 2204 may be based on physiological data of the participants and/or event related data. The animations 2204 may be selectively toggled on or off by a viewer of the event depending on whether the viewer desires to view the event with or without the animations 2204.

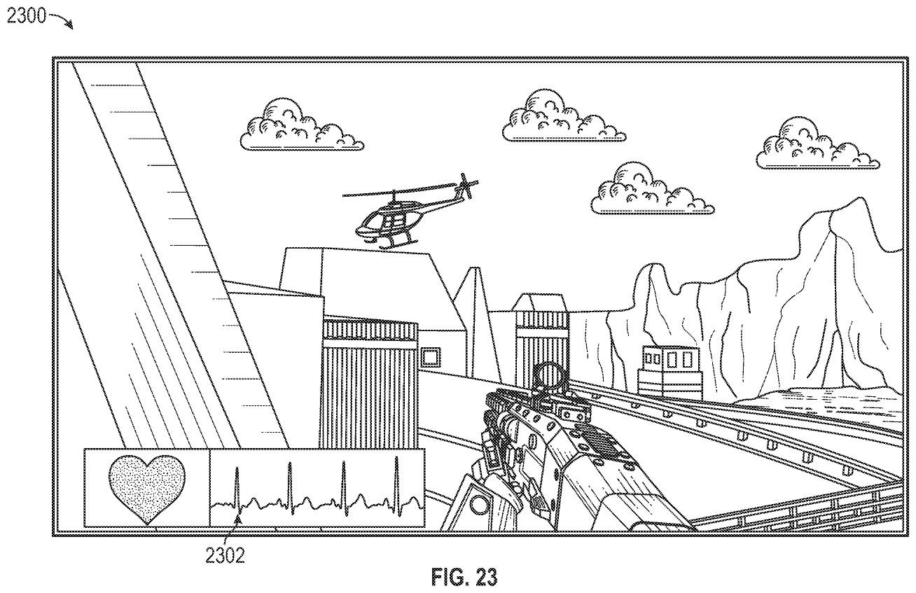

FIG. 23 illustrates an example display 2300 for displaying event related data, participant physiology related data, and the like. In this example, the event is a video game. The display 2300 may display the event to a video game player who is playing the game or to a viewer of the game who is not playing the game. The display 2300 may display physiological related data of a player of the game or a viewer of the game. In this example, the display 2300 displays cardiac related data 2302, including a heart icon and an ECG waveform. In some embodiments, the cardiac related data 2302 may be displayed to the person to whom the cardiac related data 2302 is associated. A person viewing their own physiological data may obtain feedback, in real-time, regarding their own physiological state, which may inform how they choose to play the game. In some embodiments, the cardiac related data 2302 may be displayed to a person to whom the cardiac related data 2302 is not associated such as to an opponent or teammate in the game. A person viewing, via display 2300, physiological related data of other participants in the game (e.g., opponent, teammate, etc.) may adjust their game playing techniques or strategy according to the physiological states of the other players.

In some embodiments, a player of the game may obtain points in the game based on certain physiological conditions as determined by their physiological data obtained from sensors. For example, a player may receive additional points for keeping their heart rate low during a stressful action in the game.

In some embodiments, the game may change based on a player's physiological data. For example, the game may increase or decrease in difficulty depending on a player's physiological data. As another example, a player may receive certain abilities in the game based on their physiological data. For example, an avatar of a player in a game may be able to run faster in response to certain physiological data of the player (e.g., heart rate or respiration rate exceeding a threshold). As another example, a player's shooting accuracy or ability may improve in response to the player's heart rate falling below a certain threshold. As another example, a player's avatar may move slower or see less in response to a player's SpO2 falling below a certain threshold.

In some embodiments, providing physiological data of a game participant to that game participant, in real-time, such as during a video game or virtual reality experience or augmented reality experience may condition the participant (e.g., via a visualization feedback loop) for certain physiological responses. For example, a person may wear an augmented reality or virtual reality headset in which they view and experience a stressful situation. They may also visualize their own physiological data via the headset. Visualizing their own physiological data may help them prevent undesired physiological reactions from happening such as undesirably high heart rates, respiration rates, panic attacks, and the like. Such training or conditioning may be used in settings including, military training or operations, medical training or procedures, cognitive, emotional or behavioral therapy, trauma therapy, and the like.

### ADDITIONAL EMBODIMENTS

As used herein, “real-time” or “substantial real-time” may refer to events (e.g., receiving, processing, transmitting, displaying etc.) that occur at the same time or substantially the same time (e.g., neglecting any small delays such as those that are imperceptible to humans such as delays arising from electrical conduction or transmission). As a non-limiting example, “real-time” may refer to events that occur within a time frame of each other that is on the order of milliseconds, seconds, tens of seconds, or minutes.

As used herein, “system,” “instrument,” “apparatus,” and “device” generally encompass both the hardware (for example, mechanical and electronic) and, in some implementations, associated software (for example, specialized computer programs for graphics control) components.

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

Each of the processes, methods, and algorithms described in the preceding sections may be embodied in, and fully or partially automated by, code modules executed by one or more computer systems or computer processors including computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described herein generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various features and processes described herein may be used independently of one another, or may be combined in various ways. All possible combinations and sub-combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

The various illustrative logical blocks and modules described in connection with the embodiments disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, and so forth, may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements, or blocks in the flow diagrams described herein and/or depicted in the attached figures should be understood as potentially representing modules, segments, or portions of code which include one or more executable instructions for implementing specific logical functions or steps in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

All of the methods and processes described herein may be embodied in, and partially or fully automated via, software code modules executed by one or more general purpose computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the herein-described embodiments, the elements of which are to be understood as being among other acceptable examples. All such modifications and variations are intended to be included herein within the scope of this disclosure. The section headings used herein are merely provided to enhance readability and are not intended to limit the scope of the embodiments disclosed in a particular section to the features or elements disclosed in that section. The foregoing description details certain embodiments. It will be appreciated, however, that no matter how detailed the foregoing appears in text, the systems and methods can be practiced in many ways. As is also stated herein, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Those of skill in the art would understand that information, messages, and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

What is claimed is:

1. A system for combining physiological data from a pulse oximeter coupled to a player playing a tennis match with match data corresponding to the tennis match, the system comprising:
an emitter configured to transmit optical radiation towards a tissue site of the player;
a detector configured to detect light attenuated from the tissue site responsive to the transmitted optical radiation; and
one or more hardware processors configured, via executable software instructions, to:
receive physiological data responsive to the detected light, wherein the physiological data includes one or more physiological parameters including a pulse rate;
receive match data corresponding to an occurrence in the tennis match; and
generate a visual alert to one or more viewers of the tennis match based on a trigger event corresponding to the received physiological data and the match data.

2. The system of claim 1, wherein the one or more hardware processors is further configured to generate display data for displaying, concurrently, the visual alert and a graphical representation of the event.

3. The system of claim 1, wherein the match data includes one or more of a score, a match time, a time, or player statistics.

4. The system of claim 1, wherein the trigger event is a time out, a break, a change in score, an elapsed time, a commencement of the event, a termination of the event, a score exceeding a threshold, a difference between scores exceeding a threshold, or a change in the pulse rate.

5. The system of claim 1, wherein the visual alert provides an indication of the mental state or physiological state of the player to explain player performance.

6. The system of claim 1, wherein the visual alert includes a graph or chart indicating a trend of the pulse rate.

7. The system of claim 1, wherein the visual alert includes an avatar representation of the player, wherein a color of the avatar is based on at least the pulse rate, wherein the color of the avatar is configured to change in response to a change in value of the pulse rate, and wherein the avatar is configured to perform an action based on at least the pulse rate.

8. The system of claim 1, wherein the visual alert is display to the one or more viewers, via a display, as superimposed on a ground surface of the tennis match.

9. The system of claim 1, further comprising:
a second emitter configured to transmit optical radiation towards a tissue site of a second player;
a second detector configured to detect second light attenuated from the tissue site responsive to the transmitted optical radiation;
wherein the one or more hardware processors are further configured, via the executable software instructions, to:
receive second physiological data responsive to the detected second light, wherein the second physiological data includes one or more second physiological parameters including a second pulse rate; and
update the visual alert based on a trigger event corresponding to the received second physiological data and the match data.

10. The system of claim 9, wherein the visual alert includes a graph or chart including trends of the pulse rate and the second pulse rate to provide a visual comparison of the physiological states of the player and the second player.

11. The system of claim 1, wherein the one or more hardware processors are further configured, via the executable software instructions, to:
receive historical physiological data of the player, wherein the historical physiological data includes one or more physiological parameters including a historical pulse rate, wherein the historical physiological data corresponds to physiological data gathered from the player during one or more previous tennis matches in which the player has played; and
update the visual alert based on a trigger event corresponding to the received historical physiological data and the match data.

12. The system of claim 11, wherein the visual alert includes a graph or chart including trends of the pulse rate and the historical pulse rate to provide a visual comparison of the physiological states of the player during the tennis match and the one or more previous tennis matches.

13. The system of claim 1, wherein the one or more hardware processors are further configured, via the executable software instructions, to:
store, in a database, the physiological data as historical physiological data;
store, in the database, the match data as historical match data.
access the database to retrieve the historical physiological data, wherein the visual alert is based, at least, on the historical physiological data; and
access the database to retrieve the historical match data, wherein the visual alert is based, at least, on the historical match data.

14. The system of claim 1, wherein the one or more hardware processors is configured to: generate the visual alert in response to a user request.

15. The system of claim 1, wherein the one or more hardware processors are further configured, via the executable software instructions, to:
determine, based, at least, on the physiological data and the match data, a future occurrence in the tennis match; and
determine, based, at least, on the physiological data and the match data, a probability that the future occurrence will occur.

16. The system of claim 15, wherein the future occurrence is a final match score, a change in match score, a match outcome, a winner, or a loser.

17. The system of claim 15, wherein the future occurrence is an action of the player, including at least one of scoring a point, winning the match, losing the match, breaking a record, taking a break, or making a mistake or error.

18. A method for combining physiological data from a pulse oximeter coupled to a player playing a tennis match with match data corresponding to the tennis match, the method comprising:
transmitting, via an emitter, optical radiation towards a tissue site of the player;
detecting, via a detector, light attenuated from the tissue site responsive to the transmitted optical radiation;
receiving physiological data responsive to the detected light, wherein the physiological data includes one or more physiological parameters including a pulse rate;
receiving match data corresponding to an occurrence in the tennis match; and
generating a visual alert to one or more viewers of the tennis match based on a trigger event corresponding to the received physiological data and the match data.

19. The method of claim 18, wherein the method further comprises generating display data for displaying, concurrently, the visual alert and a graphical representation of the event.

20. The method of claim 18, wherein the visual alert provides an indication of the mental state or physiological state of the player to explain player performance.
